(19) United States
(12) Patent Application Publication
(10) Pub. No.: US 2026/0007309 A1
(43) Pub. Date: Jan. 8, 2026

(54) DEVICE AND METHOD TO ASSESS RECTAL EFFLUENT

(71) Applicant: Manta Implant LLC, McLean, VA (US)

(72) Inventors: Raju S. DAVE, Gaithersburg, MD (US); Maanav ALLAMPALLAM, Potomac, MD (US); Sonya DAVEY, Gaithersburg, MD (US); Eric FRISCHE, Addison, TX (US); Ishaan JAIN, Potomac, MD (US); Gaurav SAGARWAL, GHAZIABAD (IN); Xing SU, Menlo Park, CA (US); Sudhish SWAIN, Gaithersburg, MD (US); Himanshu VERMA, McLean, VA (US); Neethi VERMA, McLean, VA (US); Sachi VERMA, McLean, VA (US)

(73) Assignee: Manta Implant LLC, McLean, VA (US)

(21) Appl. No.: 18/781,485

(22) Filed: Jul. 23, 2024

Related U.S. Application Data
(60) Provisional application No. 63/515,107, filed on Jul. 23, 2023.

(51) Int. Cl. A61B 1/31; A61B 1/00; A61B 1/06; G06T 7/00

(52) U.S. Cl. CPC A61B 1/31; A61B 1/00064; A61B 1/0684; G06T 7/0012; G06T 2207/10068; G06T 2207/30028

(57) ABSTRACT

Embodiments relate to devices, methods, and systems to assess the status of a bowel preparation in a subject. In an embodiment, the device, the methods, and the system assess the status of bowel preparation in a subject in real-time.

### INCORPORATION BY REFERENCE

This application claims priority from the U.S. Provisional Application No. 63/515,107 filed on Jul. 23, 2023, the content of which are incorporated herein by reference in their entirety.

### FIELD OF THE INVENTION

The present disclosure relates to systems and methods for assessing rectal effluent. In particular, the present disclosure relates to systems and methods to determine the status of colon cleansing in subjects prior to a gastrointestinal procedure.

### BACKGROUND

“GI diseases contribute substantially to health care use in the United States. Total expenditures for GI diseases are $135.9 billion dollars annually—greater than for other common diseases. Expenditures are likely to continue increasing.” [Source: Perry et al., Burden and Cost of Gastrointestinal, Liver, and Pancreatic Diseases in the United States: Update 2018. Gastroenterology, 2019 January; 156 (1): 254-272.e11. doi: 10.1053/j.gastro.2018.08.063]

“Colorectal cancer is the third most common cancer in the USA. Colonoscopy is considered the gold standard for colorectal cancer screening and can offer both diagnosis and therapy. The bowel preparation remains a significant barrier for patients who need to undergo colonoscopy and is often cited as the most dreaded aspect of the colonoscopy process. Inadequate bowel preparations still occur in 10-25% of colonoscopies, and this in turn can lead to increased procedural times, lower cecal intubation rates, and shorter interval between colonoscopies. From a quality standpoint, it is imperative that we do what we can to decrease the rate of inadequate bowel preparations.” [Source: Millien V O, Mansour N M. Bowel Preparation for Colonoscopy in 2020: A Look at the Past, Present, and Future. Curr Gastroenterol Rep. 2020 May 6; 22 (6): 28. doi: 10.1007/s11894-020-00764-4. PMID: 32377915.]

“The cost of a colonoscopy will vary greatly between inpatient and outpatient facilities. The national average cost for a colonoscopy at inpatient facilities is $4,350, while the same procedure at outpatient facilities averaged $2,550.” [Source: How much does a colonoscopy cost? Colonoscopy, Cost of Medical Procedures in the U.S., Gastroenterology]

“Decreased incidence of colon cancer is associated with optimal colonoscopic examination of the entire colon. Regardless of indication, the success of colonoscopy is closely related to adequate colon preparation. However, it has been reported that inadequate bowel cleaning is observed in approximately 25% of all colonoscopies. Adverse results of insufficient colon cleaning include decreased adenoma detection and cecal intubation rates, prolonged procedural times, and shortened surveillance intervals.” [Source: Sim J S, Koo J S. Predictors of Inadequate Bowel Preparation and Salvage Options on Colonoscopy. Clin Endosc. 2016 July; 49 (4): 346-9. doi: 10.5946/ce.2016.094. Epub 2016 Jul. 29. PMID: 27484811; PMCID: PMC4977741.]

“Current colonoscopy prep medications are dosed “one size fits all.” However, patients with certain risk factors, including but not limited to a history of poor colon prep on previous colonoscopies, male gender, obesity, hypertension, diabetes, constipation, drags that affect motility (narcotics, tri-cyclic antidepressants), and low educational level, are at higher risk of having an inadequate or suboptimal prep. Overall, approximately 40-70% of all colonoscopy patients have at least one recognizable risk factor for a suboptimal prep. Unfortunately, no one has been able to construct a schema to allow individualized dosing of the prep with any useful accuracy.” [WO2021146420A1 titled “Systems and methods for assessing colonoscopy preparation” published on Jul. 22, 2021.]

“The quality of bowel preparation can be affected by many patient factors, including poor sociodemographic status, presence of comorbidities, certain medications and the timing of colonoscopy. These factors account for approximately 25% of inadequate bowel preparations (1, 2, 5, 6). With approximately 11 million colonoscopies performed just in 2013 we can acknowledge the significant healthcare and economic burden of inadequate preparations, which can lead to missed colon cancers, repeat procedures and potentially prolonged hospitalizations” [Source: Agrawal et. al, Predictors of poor bowel preparations and colonoscopy cancellations in inpatient colonoscopies, a single center retrospective study. Transl Gastroenterol Hepatol 2022; 7:4.]

“Patients' description of last rectal effluent is not a reliable predictor of quality of preparation per the endoscopist, but patients reporting their last effluent as brown liquid or solid have a substantial likelihood of inadequate preparation. These patients may benefit from additional preparation, which may be particularly useful if it can be administered in the endoscopy unit followed by colonoscopy on the same day.” [Fatima H, Johnson C S, Rex D K. Patients' description of rectal effluent and quality of bowel preparation at colonoscopy. Gastrointest Endosc. 2010 June; 71 (7): 1244-1252.e2. doi: 10.1016/j.gie.2009.11.053. Epub 2010 Apr. 1. PMID: 20362286.]

Therefore, there is a pressing need for a smart device, system, or method to accurately evaluate the state of colon cleansing in patients prior to a gastrointestinal procedure. Such a solution could significantly enhance the effectiveness of colonoscopies, potentially reducing healthcare expenses and improving patient outcomes.

### SUMMARY

The described devices, systems and methods addresses the pressing problem of inadequate bowel preparation, reduces healthcare expenses, and improves patient outcomes. The system evaluates the state of bowel preparation in a patient in real-time and provide insights to improve the success rate of colonoscopy. The invention aims to identify patients who are not adequately prepared for colonoscopy. The expected accuracy of the proposed smart colonoscopy preparation system is 99%.

An embodiment relates to a method comprising providing a reference background for measuring a plurality of parameters, capturing an image of rectal effluent along with the reference background, assessing the image for readiness level of bowel preparation, estimating the readiness level by measuring the plurality of parameters, and providing an assessment of bowel preparation to undergo a medical procedure. In an embodiment, the reference background comprises a reticle. In another embodiment, the reticle is used for measuring one or more of the plurality of parameters. In yet another embodiment, the measuring comprises one or more of a color measurement, a diameter measurement of a microscopic object, a diameter measurement of the microscopic object, a thickness measurement of the microscopic object, calibration and alignment. In yet another embodiment, the plurality of parameters comprises two or more of color, turbidity, particulate concentration, size distribution, stickiness, and viscosity. In yet another embodiment, the image comprises one or more images and videos. In yet another embodiment, the image comprises a pre bowel image and a post bowel image. In yet another embodiment, assessing comprises analysis of the image. In yet another embodiment, the analysis of the image comprises producing a digital representation of the image, subtracting the pre bowel image and the post bowel image to generate a subtracted image, classifying objects in the digital representation based on a detectable feature, secondarily classifying cells having the detectable feature atypical of unexpected in a normal image among the objects identified in a classification step using adaptive processing, and generating one or more classifications output comprising particle size and color distribution of the subtracted image. In yet another embodiment, the method further comprising outputting the assessment of the bowel preparation is based on the one or more classifications output by a trained neural network. In yet another embodiment, the trained neural network is generated by training a neural network for the feature detection comprising obtaining one or more training data of the pre bowel image and the post bowel image, generating at least one feature map from the training data, outputting prediction data of the feature map, based on one or more predefined categories, and tuning a parameter applied to the network, based on the prediction data, wherein the training is repeatedly performed until preset termination conditions are satisfied. In yet another embodiment, the one or more training data comprises a generative adversarial network generated image. In yet another embodiment, the estimating comprises providing a score based on Boston Bowel Preparation Scale (BBPS). In yet another embodiment, the score is one of a 0, 1, 2, and 3. In yet another embodiment, the readiness level comprises a cleanliness class comprising one of an unacceptable colon state, a poor colon state, a fair colon state, and a good colon state. In yet another embodiment, providing the assessment comprises generating an indication via at least a visual indication and an auditory indication of the score. In yet another embodiment, the medical procedure comprises colonoscopy. In yet another embodiment, the method further comprises providing a recommendation for actions to be taken based on the assessment to at least one of a health provider and a subject.

In yet another embodiment, the method further comprising, providing a recommendation based on at least two of the one or more classifications output, a viscosity measurement, a stickiness measurement, data obtained from one or more sensors, patient factors and procedural factors. In yet another embodiment, the recommendation can be one or more of go ahead with the medical procedure, one or more medical tests, a time interval and a laxative dose before the medical procedure.

An embodiment relates to a device comprising a disposable assembly comprising an adjustable frame and an endoscope, and a reusable assembly comprising a detachable Universal Serial Bus port cable configured to connect with an external device. In an embodiment, the device is affixable to a toilet commode. In another embodiment, the adjustable frame comprises a 3D target for aiding in focusing and is configured for providing sharp and clear image. In yet another embodiment, the 3D target comprises a reticle. In yet another embodiment, the reticle acts as a reference point for measuring one or more of a color measurement, a diameter measurement of a microscopic object, a thickness measurement of the microscopic object, calibration and alignment. In yet another embodiment, the endoscope is configured to capture an image of a suspension in a commode. In yet another embodiment, the endoscope comprises a light emitting diode. In yet another embodiment, the external device is configured for sending a command to the device for capturing an images comprising the 3D target. In yet another embodiment, the detachable Universal Serial Bus port cable comprises a connector and a port. In yet another embodiment, the detachable Universal Serial Bus port cable is used for power supply and data transfer.

An embodiment relates to a device comprising a flat rod comprising a first end and a second end, the first end configured to act as a hook for stability and the second end comprising a camera for image acquisition. In an embodiment, the hook is configured for positioning the device securely onto a toilet commode. In another embodiment, the second end comprises a miniature camera module and a light emitting diode illumination for optimal imaging. In yet another embodiment, the second end is configured for capturing a high-resolution image of a suspension in the toilet commode. In yet another embodiment, the second end submerged in the suspension during use. In yet another embodiment, the device is configured to send the high-resolution image to an external device. In yet another embodiment, the external device is one of a mobile phone, a tablet, an iPad, and a computing device. In yet another embodiment, the flat rod is sturdy, durable, and lightweight. In yet another embodiment, the flat rod is made up of one of an alloy, a metal, and a polymer. In yet another embodiment, the first end comprises a Universal Serial Bus port.

An embodiment relates to a system comprising an image capturing unit comprising a reference background configured to be attached to a toilet commode, a communication module, a processor and a non-transitory storage medium comprising instructions that, when executed, causes the processor to capturing an image of a rectal effluent along with the reference background, assessing the image for readiness level of bowel preparation, estimating the readiness level by measuring a plurality of parameters, and providing an assessment of the bowel preparation to undergo a medical procedure. In an embodiment, the reference background comprises a reticle. In another embodiment, the reticle for the measuring. In yet another embodiment, the measuring comprises one or more of a color measurement, a diameter measurement of a microscopic object, a diameter measurement of the microscopic object, a thickness measurement of the microscopic object, calibration and alignment. In yet another embodiment, the plurality of parameters comprises two or more of color, turbidity, particulate concentration, size distribution, stickiness, and viscosity. In yet another embodiment, the system further comprising a sampling unit to collect samples of the rectal effluent for further analysis. In yet another embodiment, the system further comprising a sensor to measure a biomarker in the rectal effluent. In yet another embodiment, the biomarker is one of a diagnostic biomarker, a prognostic biomarker, and a monitoring biomarker. In yet another embodiment, the system further comprising an indicator configured for generating an indication via at least a visual indication and an auditory indication for image capturing and successful image capturing.

An embodiment relates to a device comprising a port for phone attachment, and an optical tube and wherein the device is affixable to a toilet commode, and wherein the device can capture images of suspension collected in the toilet commode. In an embodiment, the device can be operated via an external device. In another embodiment, the device is connected with the external device via one of a wired connection, and a wireless connection. In yet another embodiment, the device further comprises a Universal Serial Bus port cable. In yet another embodiment, Universal Serial Bus port cable is used for power supply. In yet another embodiment, the Universal Serial Bus port cable is used for data transfer.

An embodiment relates to a device comprising a built-in camera and an optical tube and wherein the device is affixable to a toilet commode, and wherein the built-in camera can capture images of suspension collected in the toilet commode. In an embodiment, the device is connected with an external device via one of a wired connection, and a wireless connection. In another embodiment, a setting for the built-in camera is adjusted via the external device.

An embodiment relates to a device comprising a port for phone attachment, an optical tube, and a stirrer and wherein the device is affixable to a toilet commode, and wherein the device can capture images of suspension collected in the toilet commode.

An embodiment relates to a device comprising a built-in camera, an optical tube, and a stirrer and wherein the device is to a toilet commode, and wherein the built-in camera can capture images of suspension collected in the toilet commode.

An embodiment relates to a device comprising a sample funnel, and a sample container comprising an analysis cell, and wherein the sample funnel attaches to the sample container from beneath at an open end of the sample container. In an embodiment, the sample funnel is configured to channel a rectal effluent to the sample container. In another embodiment, the sample funnel is made up of a flushable material. In yet another embodiment, the flushable material comprises cellulose fibers and a water insoluble film to increase tensile strength of the flushable material. In yet another embodiment, the sample funnel is one of a bungee type sample funnel and an adhesive type sample funnel. In yet another embodiment, the adhesive type sample funnel comprises a waterproof adhesive. In yet another embodiment, the waterproof adhesive comprises one or more of a water-based emulsion adhesives, an acrylic rubber-based adhesive, and a cross-link coating. In yet another embodiment, the sample funnel is made up of a biodegradable material. In yet another embodiment, the sample funnel is configured with varying levels of thickness at different locations to conserve materials and increase strength. In yet another embodiment, the sample funnel comprises a curtain coating. In yet another embodiment, the sample funnel comprises a bilayer film. In yet another embodiment, the sample funnel comprises one of a waterproof adhesive and a cross linking to seal. In yet another embodiment, the analysis cell comprises a single cell fill, a matte white sample background and a matte white calibration surface. In yet another embodiment, the analysis cell comprises the matte white sample background at a close end of the sample container. In yet another embodiment, a reticle is printed on the matte white sample background. In yet another embodiment, the single cell fill comprises a float. In yet another embodiment, the float is a gel-based float. In yet another embodiment, the float is optically clear. In yet another embodiment, the single cell fill is configured to be fill only once. In yet another embodiment, the sample container is made up of one of a cellulose material and Polylactic acid (PLA) material. In yet another embodiment, a first portion of the sample funnel is open and is configured to allow most of urine to bypass entering the sample container. In yet another embodiment, a slanting wall is added to a second portion near to the sample container to only allow rectal effluent to enter the analysis cell. In yet another embodiment, the device comprises an identification number. In yet another embodiment, the matte white sample background and the matte white calibration surface for calibrating an image captured by a camera. In yet another embodiment, the reticle assists in measurements and precise analyses. In yet another embodiment, the device further comprising one or more sensors to measure medically relevant rectal effluent parameters. In yet another embodiment, the medically relevant rectal effluent parameters comprise one or more of color, turbidity, particle size, stickiness, viscosity, and distribution accuracy. In yet another embodiment, the one or more sensors are one of a line sensor, a spot sensor and a combination there off. In yet another embodiment, the device is made up of a biodegradable material. In yet another embodiment, the device is made up of a flushable material.

An embodiment relates to a device comprising a stirrer configured to homogenize a rectal effluent, an imaging portion configured to capture an image of the rectal effluent, and a communication unit configured to transmit the image to a user system. In an embodiment, a speed of the stirrer is adjustable. In another embodiment, the speed is adjusted manually using a knob or a dial on the stirrer. In yet another embodiment, the speed is adjusted using a programmable logic controller. In yet another embodiment, the speed of the stirrer is adjusted as necessary to reduce a frothing condition. In yet another embodiment, the imaging portion comprises an illumination unit configured to emit illumination light with which the rectal effluent is irradiated, an image sensor to receive light from the rectal effluent, perform photoelectric conversion of the received light, and an optical sensor configured to measure amount of the illumination light emitted by the light source. In yet another embodiment, the imaging portion further comprises an illumination controller configured to control illumination. In yet another embodiment, the device is affixable to a toilet commode. In yet another embodiment, the stirrer comprises sensors to detect frothing. In yet another embodiment, the device is configured to generate an alert if frothing occurs beyond a set threshold.

An embodiment relates to a device comprising a stirrer configured to homogenize a rectal effluent, wherein the stirrer comprises a sampling port and a container, and wherein the device is configured to obtain the rectal effluent. In an embodiment, the stirrer is connected to a motor unit. In another embodiment, the motor unit comprises an electrical drive motor. In yet another embodiment, the electrical drive motor is coupled to and arranged for driving a motor drive shaft of the motor unit. In yet another embodiment, the electrical drive motor is powered by a battery. In yet another embodiment, the electrical drive motor is powered by a main electrical line. In yet another embodiment, a rotation speed of the stirrer is adjustable via the motor unit. In yet another embodiment, the stirrer is removably connected to the device via a port. In yet another embodiment, the stirrer comprises sensors to detect frothing. In yet another embodiment, the device is configured to generate an alert if frothing occurs beyond a set threshold.

An embodiment relates to a device comprising a first portion comprising an imaging portion that is configured to capture an image of a rectal effluent, and a second portion comprising a stirrer, a sampling port, and a container wherein the second portion is configured to obtain the rectal effluent. In an embodiment, the device is configured to determine a status of colon cleansing in a subject. In another embodiment, the first portion is re-usable. In yet another embodiment, the second portion is disposable. In yet another embodiment, the stirrer is connected to a motor unit. In yet another embodiment, the motor unit comprises an electrical drive motor. In yet another embodiment, the electrical drive motor is coupled to and arranged for driving a motor drive shaft of the motor unit. In yet another embodiment, the electrical drive motor is powered by a battery. In yet another embodiment, the electrical drive motor is powered by a main electrical line. In yet another embodiment, the stirrer is used to homogenize the rectal effluent. In yet another embodiment, a rotation speed of the stirrer is adjustable via the motor unit. In yet another embodiment, the stirrer is removably connected to the device via a port. In yet another embodiment, the stirrer comprises sensors to detect frothing. In yet another embodiment, the device is configured to generate an alert if frothing occurs beyond a set threshold. In yet another embodiment, the device is configured to eject an anti-frothing agent if the frothing occurs beyond a set threshold. In yet another embodiment, the stirrer is made up of a biodegradable polymer. In yet another embodiment, the biodegradable polymer is one of a cellulose, poly lactic acid polymer and Poly (3-hydroxybutyrate-co-3-hydroxyvalerate). In yet another embodiment, the stirrer comprises micro coils that are configured for collecting solid rectal effluent. In yet another embodiment, the stirrer is foldable to fit in a test tube for further analysis of rectal effluent. In yet another embodiment, sampling through the sampling port can be done using one of an autosampler, a grab sampler and a thief sampler. In yet another embodiment, the device can be operated via a remote control. In yet another embodiment, the device can be operated through an external device via an app. In yet another embodiment, the external device can be one of a laptop, a palm top, a mobile phone, a tablet, and a desktop computer. In yet another embodiment, the device is connected to the external device via a connectivity interface. In yet another embodiment, the connectivity interface is a wireless link. In yet another embodiment, the wireless link is at least one of a Bluetooth, a Wi-Fi, and a hotspot. In yet another embodiment, the connectivity interface is a wired link. In yet another embodiment, the image comprises a plurality of images. In yet another embodiment, the device is integrated into an information processing module. In yet another embodiment, the information processing module analyzes data from the plurality of images of the rectal effluent. In yet another embodiment, the information processing module is a tablet computer, a portable computer, or a wearable computer. In yet another embodiment, the device further comprises a colorimetric device to measure color of the rectal effluent. In yet another embodiment, the device further comprises a turbidimeter to measure turbidity of the rectal effluent from the image. In yet another embodiment, the device further comprises a nephelometer for measuring a concentration of suspended particulates in the rectal effluent. In yet another embodiment, the device is releasably attachable to a toilet commode. In yet another embodiment, the toilet commode is a squat type toilet commode. In yet another embodiment, the toilet commode is a sitting type toilet commode. In yet another embodiment, the device provides an indication of status of colon prep. In yet another embodiment, the device comprises a central electronic unit that is coupled to a first electronic unit of the first portion and a second electronic unit of the second portion of the device. In yet another embodiment, the central electronic unit comprises a plurality of switches for manual operation. In yet another embodiment, the central electronic unit is connected to a processor and a memory. In yet another embodiment, the memory is in electronic communication with the processor. In yet another embodiment, the memory comprises programming code for execution by the processor. In yet another embodiment, the programming code comprises codes for changing a speed of the stirrer if frothing occurs beyond a set threshold. In yet another embodiment, the processor is configured to analyze the captured image and generate an alert when analysis indicates completion of colon preparation. In yet another embodiment, the processor is further configured to take a sample of rectal effluent using the second portion when a command is provided. In yet another embodiment, the central electronic unit comprises a cyber security element configured for secured information transaction.

An embodiment relates to a method comprising obtaining a rectal effluent using a device, capturing an image of the rectal effluent, assessing the image for readiness level of bowel preparation, and providing an assessment of the bowel preparation to undergo colonoscopy, and wherein the device comprises a first portion comprising an imaging portion configured to captures an image of the rectal effluent and a second portion comprising a stirrer, a suction pump, and a container, wherein the second portion is configured to obtain the rectal effluent. In an embodiment, the method further comprises comparing the image captured with a pre-loaded image and providing an indication of a status of colon preparation. In another embodiment, the assessment is done using pixel analysis. In yet another embodiment, the assessment is done using a machine learning-based model. In yet another embodiment, the method further comprises sending the image captured to a healthcare provider for feedback.

An embodiment relates to a system comprising a device comprising a first portion comprising an imaging portion configured to captures an image of rectal effluent, and a second portion comprising a stirrer, a suction pump, and a container wherein the second portion is configured to obtain the rectal effluent, a communication module configured to communicate with a computing device, and the computing device comprising a processor and a tangible non-transitory memory, configured to communicate with the processor, wherein the tangible non-transitory memory has stored thereon instructions executable by the system to cause the system to perform operations comprising receive a captured image through a receiving component convert intensity data from at least one of a red channel, a green channel, or a blue channel from at least a portion of the image to a first data point having a first value through a pre-processing module, compare the first data point with a plurality of data points from a standardized curve to identify a value of an image-based assay parameter via an information processing module, predict a cleanliness class and readiness level based on the value of the assay parameter via an information processing module, and provide a recommendation based on the cleanliness class and the readiness level.

In an embodiment, the system further comprises a sensor. In another embodiment, the system is further operable to receive data collected via one or more sensors through the receiving component, processing of the data collected via the one or more sensors to filter a noise present in the data and get a normalized dataset, compare the normalized dataset with a standardized model to identify a second value of a sensor-based assay parameter via the information processing module, correlate the first value with the second value, and generate a percentage level for the cleanliness class and the readiness level. In yet another embodiment, the sensor comprises at least one of a colorimeter, spectrophotometer and a turbidimeter. In yet another embodiment, the computing device is remotely located. In yet another embodiment, a machine learning module is configured to train a machine learned model that is leveraged by the system. In yet another embodiment, the machine learning module is configured to train a rules-based recommendation system. In yet another embodiment, the system uses supervised deep learning for automated feature learning from raw training images for assessment of the readiness level of colon preparation. In yet another embodiment, a machine learning model is configured to learn using labelled data using a supervised learning method, wherein the supervised learning method comprises logic using at least one of a decision tree, a logistic regression, a support vector machine, a k-nearest neighbors, a Naïve Bayes, a random forest, a linear regression, a polynomial regression, and a support vector machine for regression. In yet another embodiment, the machine learning model is configured to learn from a real-time data using an unsupervised learning method, wherein the unsupervised learning method comprises logic using at least one of a k-means clustering, a hierarchical clustering, a hidden Markov model, and an apriori algorithm. In yet another embodiment, the machine learning model has a feedback loop, wherein an output from a previous step is fed back to the machine learning model in real-time to improve the performance and accuracy of the output of a next step. In yet another embodiment, the machine learning model comprises a recurrent neural network model. In yet another embodiment, the machine learning model has a feedback loop, wherein a learning is further reinforced with a reward for each true positive of an output of the system. In yet another embodiment, the system further comprises a cyber security module.

An embodiment relates to a device comprising a first portion comprising an imaging portion configured to captures an image of the rectal effluent, a second portion comprising a stirrer, a sampling port, and a container wherein the second portion is configured to obtain the rectal effluent, and a sensor.

An embodiment relates to a device comprising a first portion comprising an imaging portion configured to captures an image of a rectal effluent, a second portion comprising a stirrer, a sampling port, and a container wherein the second portion is configured to obtain the rectal effluent, and a colorimeter.

An embodiment relates to a device comprising a first portion comprising an imaging portion configured to captures an image of a rectal effluent, a second portion comprising a stirrer, a sampling port, and a container wherein the second portion is configured to obtain the rectal effluent, and a spectrophotometer.

An embodiment relates to a device comprising a first portion comprising an imaging portion configured to captures an image of a rectal effluent, a second portion comprising a stirrer, a sampling port, and a container wherein the second portion is configured to obtain the rectal effluent, and a turbidimeter.

### BRIEF DESCRIPTION OF THE FIGURES

The patent or application file contains at least one drawing executed in color. Copies of this patent or patent application publication with color drawing(s) will be provided by the Office upon request and payment of the necessary fee. In the present disclosure, reference is made to the accompanying drawings, which form a part hereof. In the drawings, similar symbols typically identify similar components, unless context dictates otherwise. Various embodiments described in the detailed description, drawings, and claims are illustrative and not meant to be limiting. Other embodiments may be used, and other changes may be made, without departing from the spirit or scope of the subject matter presented herein. It will be understood that the aspects of the present disclosure, as generally described herein, and illustrated in the Figures, can be arranged, substituted, combined, separated, and designed in a wide variety of different configurations, all of which are contemplated herein.

FIG. 1A illustrates a method for assessing readiness of colon preparation, according to an embodiment.

FIG. 1B provides a pictorial presentation of method for assessing readiness of colon preparation, according to one or more embodiments.

FIG. 2A shows schematic a colonoscopy readiness device, according to one or more embodiments.

FIG. 2B shows example views of the target, according to one or more embodiments.

FIG. 2C shows a pictorial representation attaching the device to a commode, according to one or more embodiments.

FIG. 3 illustrates a colonoscopy readiness device, according to one or more embodiments.

FIG. 4 illustrates a flowchart for using the device described in the various embodiments.

FIG. 5A illustrates a device with optical tube and an adaptor for holding a camera, according to one or more embodiments.

FIG. 5B illustrates a device with optical tube and built-in camera according to one or more embodiments.

FIG. 5C and FIG. 5D illustrate a device with optical tube, built-in camera and a stirrer, according to one or more embodiments.

FIG. 5E illustrates a device comprising a stirrer, according to one or more embodiments.

FIG. 6A illustrates a flushable device designed to be attached to a toilet commode, according to one or more embodiments.

FIG. 6B illustrates (i) bungee type and (ii) adhesive type of device, according to one or more embodiments.

FIG. 6C illustrates a transparent view of sample container of the flushable device, according to one or more embodiments.

FIG. 6D illustrates reticle options that can be used in the device, according to one or more embodiments.

FIG. 6E illustrates a partial view of disposable seat cover, according to one or more embodiments.

FIG. 6F illustrates a bottom view and a cross-sectional view of the analysis cell from different angles.

FIG. 6G illustrates an analysis cell comprising a reticle, according to one or more embodiments.

FIG. 6H shows a stickiness indicator and a viscosity indicator of the device, according to one or more embodiments.

FIG. 7A illustrates a colonoscopy readiness device, according to one or more embodiments.

FIG. 7B illustrates a top view and a bottom view of the electronics unit of the colonoscopy readiness device, according to one or more embodiments.

FIG. 7C illustrates a cutaway of the fluid container of the device, according to one or more embodiments.

FIG. 7D illustrates a mechanism of bowel fluid flow through the device, according to one or more embodiment.

FIG. 7E illustrates viscosity and density measurement mechanism in the device, according to one or more embodiments.

FIG. 7F illustrates a device comprising a biome vacuum sample jar, according to one or more embodiments.

FIG. 8A illustrates a system comprising a colonoscopy readiness device, according to one or more embodiments.

FIG. 8B illustrates a system comprising the colonoscopy readiness device, according to an embodiment.

FIG. 8C illustrates a system comprising the colonoscopy readiness device, according to an embodiment.

FIG. 8D illustrates a system comprising the colonoscopy readiness device, according to an embodiment.

FIG. 9 illustrates a method for assessing readiness of colon preparation, according to an embodiment.

FIG. 10 describes a table for scale and readiness analysis via colonoscopy readiness device, according to an embodiment.

FIG. 11 is an exemplary block diagram illustrating some of the components of the exemplary system.

FIG. 12 is a schematic diagram illustrating one embodiment of a system including an image capture device with a wireless transmitter/receiver.

FIG. 13 illustrates a block diagram of the system according to one or more embodiments.

FIG. 14 is a flowchart of an exemplary process for completing a colon assessment according to embodiments of the present disclosure.

FIG. 15A shows a structure of the neural network/machine learning model with a feedback loop.

FIG. 15B shows a structure of the neural network/machine learning model with reinforcement learning.

FIG. 15C shows a workflow for analyzing bowel preparation quality using a combination of image processing techniques and neural networks.

FIG. 15D illustrates image processing for it shows imaging and analysis process using Convolutional Neural Networks (CNNs) and Recurrent Neural Networks (RNNs) for bowel preparation analysis.

FIG. 15E depicts a process for training a neural network to access readiness level of the colon by analyzing an image of a rectal effluent.

FIG. 15F illustrates a process for generating training images. FIG. 15G shows learning process of a Convolutional Neural Network (CNN) to recognize and classify features in images.

FIG. 15H shows CNN architecture for image recognition.

FIG. 16 shows a block diagram of the cyber security module in view of the system and server.

### DETAILED DESCRIPTION

### Definitions and General Techniques

For simplicity and clarity of illustration, the figures illustrate the general manner of construction. The descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denotes the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The terms “couple,” “coupled,” “couples,” “coupling,” and the like should be broadly understood and refer to connecting two or more elements mechanically and/or otherwise. Two or more electrical elements may be electrically coupled together, but not mechanically or otherwise coupled together. Coupling may be for any length of time, e.g., permanent or semi-permanent or only for an instant. “Electrical coupling” and the like should be broadly understood and include electrical coupling of all types. The absence of the word “removably”, “removable”, and the like near the word “coupled” and the like does not mean that the coupling, etc. in question is or is not removable.

As defined herein, two or more elements are “integral” if they are comprised of the same piece of material. As defined herein, two or more elements are “non-integral” if each is comprised of a different piece of material.

As defined herein, “real-time” can, in some embodiments, be defined with respect to operations carried out as soon as practically possible upon occurrence of a triggering event. A triggering event can include receipt of data necessary to execute a task or to otherwise process information. Because of delays inherent in transmission and/or in computing speeds, the term “real time” encompasses operations that occur in “near” real time or somewhat delayed from a triggering event. In a number of embodiments, “real time” can mean real time less a time delay for processing (e.g., determining) and/or transmitting data. The particular time delay can vary depending on the type and/or amount of the data, the processing speeds of the hardware, the transmission capability of the communication hardware, the transmission distance, etc. However, in many embodiments, the time delay can be less than approximately one second, two seconds, five seconds, or ten seconds.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in art.

The methods and techniques of the present invention are generally performed according to conventional methods well known in the art and as described in various general and more specific references that are cited and discussed throughout the present specification unless otherwise indicated. The nomenclatures used in connection with, and the procedures and techniques of, the embodiments herein, and other related fields described herein, are those well-known and commonly used in art.

The following terms and phrases, unless otherwise indicated, shall be understood to have the following meanings.

The term “rectal effluent” or “bowel fluid” as used herein refers to excretion output by a subject. It is usually liquid in texture. In some embodiments, it is semi liquid in texture. For some embodiments of the device, the rectal effluent can be one of a solid, liquid or semi liquid in texture.

The term “bowel preparation” or “bowel cleansing” or “colon readiness” or “colon cleansing” refers to a method for flushing out the large intestine. It is a medical technique to bowel cleanse to prepare for colonoscopy or colorectal surgery. Colon cleansing is done using powdered supplements, liquid supplements or colon irrigation.

The term “subject” refers to a person or patient going through bowel cleansing procedure.

The term “toilet commode” refers to a piece of sanitary hardware that collects human urine and feces, and sometimes toilet paper, usually for disposal. The example of toilet commodes are squat type toilet commode and toilet sitting type commode.

The term “colonoscopy” or “coloscopy” refers to an endoscopic examination of the large bowel and the distal part of the small bowel with a CCD camera or a fiber optic camera on a flexible tube passed through the anus. It can provide a visual diagnosis (e.g., ulceration, polyps) and grants the opportunity for biopsy or removal of suspected colorectal cancer lesions.

The term “virtual colonoscopy” or “VC” refers to a new method of screening the colon for precancerous polyps. Using a computed tomography (CT) scanner and new computer methods of rendering or reconstructing the images, the colon can be evaluated without a colonoscope and without sedation. After cleansing, the colon is distended with air or carbon dioxide and the CT scan is performed in seconds. The patient is scanned both on his stomach as well as on his back to allow excess fluid and stool to fall away from the dependent portion of the colon.

As used herein “reticle”, is a include a network of colored lines or patterns that are placed on a matt background and serve as a measuring scale or an aid in locating and focusing on objects. The colors in the reticle enhance the functionality of the reticle by enabling color matching through image analysis. This is particularly useful in applications where precise color differentiation is needed. In an embodiment, a reticle design is provided comprising colored circles/patterns for contrast and for precise color differentiation using image analysis.

As referred herein, “color space” is a specific organization of colors. In combination with color profiling supported by various physical devices, it supports reproducible representations of color-whether such representation entails an analog or a digital representation. The RGB color space represents images as an m-by-n-by-3 numeric array whose elements specify the intensity values of the red, green, and blue color channels. The HSV (Hue, Saturation, Value) color space corresponds better to how people experience color than the RGB color space does. CIELAB 1976 XYZ and CIELAB 1976 L*a*b* are device-independent color spaces developed by the International Commission on Illumination, known by the acronym CIE. Color spaces model colors according to the typical sensitivity of the three types of cone cells in the human eye.

As referred herein, “colorimeter” is an instrument for measuring the absorbance of particular wavelengths of light by a specific solution. Colorimeters are instruments that quantify concentration via absorbance measurements using spectrophotometry and Beer-Lambert law.

As referred herein, “turbidimeter” is an instrument for measuring the loss of intensity of transmitted light due to the scattering effect of particles suspended in it. The turbidimeter may comprise fiber optic sensors for increased sensitivity over large concentration ranges, and fiber optic cable to allow online measurements.

As referred herein, “nephelometer” is an instrument for measuring the concentration of suspended particulates in a liquid or gas colloid. A nephelometer measures suspended particulates by employing a light beam (source beam) and a light detector set to one side of the source beam. Particle density is then a function of the light reflected into the detector from the particles.

As referred herein, “line sensor” is a type of sensor or detector that is designed to identify specific markings, lines, or patterns on the cellulose sheet, typically by utilizing optical or electrical technology.

As referred herein, “spot sensor” is a type of sensor that is designed to detect or measure specific points or spots on an object or surface.

The term “alert” as used herein refers to an audio signal, visual signal, or other sensory signal or the like for a subject.

The term “removably connected” means that the device component can be connected to, and disconnected from, the device by a suitable method (e.g. clockwise and anti-clockwise movement, push-in and pull-out).

As used herein “Machine learning” refers to algorithms that give a computer the ability to learn without being explicitly programmed, including algorithms that learn from and make predictions about data. Machine learning algorithms include, but are not limited to, decision tree learning, artificial neural networks (ANN) (also referred to herein as a “neural net”), deep learning neural network, support vector machines, rules-based machine learning, random forest, etc. For the purposes of clarity, algorithms such as linear regression or logistic regression can also be used as part of a machine learning process. However, it is understood that using linear regression or another algorithm as part of a machine learning process is distinct from performing a statistical analysis such as regression with a spreadsheet program. The machine learning process can continually learn and adjust the classifier as new data becomes available and does not rely on explicit or rules-based programming. The ANN may be featured with a feedback loop to adjust the system output dynamically as it learns from the new data as it becomes available. In machine learning, backpropagation and feedback loops are used to train the AI/ML model, improving the model's accuracy and performance over time.

The term “cyber security” as used herein refers to application of technologies, processes, and controls to protect systems, networks, programs, devices, and data from cyber-attacks.

The term “cyber security module” as used herein refers to a module comprising application of technologies, processes, and controls to protect systems, networks, programs, devices and data from cyber-attacks and threats. It aims to reduce the risk of cyber-attacks and protect against the unauthorized exploitation of systems, networks, and technologies. It includes, but is not limited to, critical infrastructure security, application security, network security, cloud security, Internet of Things (IoT) security.

The term “encrypt” used herein refers to securing digital data using one or more mathematical techniques, along with a password or “key”, to decrypt the information. It refers to converting information or data into a code, especially to prevent unauthorized access. It may also refer to concealing information or data by converting it into a code. It may also be referred to as cipher, code, encipher, encode. A simple example is representing alphabets with numbers-say, ‘A’ is ‘01’, ‘B’ is ‘02’, and so on. For example, a message like “HELLO” will be encrypted as “0805121215,” and this value will be transmitted over the network to the recipient(s).

The term “decrypt” used herein refers to the process of converting an encrypted message back to its original format. It is generally a reverse process of encryption. It decodes the encrypted information so that only an authorized user can decrypt the data because decryption requires a secret key or password. This term could be used to describe a method of unencrypting the data manually or unencrypting the data using the proper codes or keys.

The term “cyber security threat” used herein refers to any possible malicious attack that seeks to unlawfully access data, disrupt digital operations, or damage information. A malicious act includes, but is not limited to, damaged data, stolen data, or disrupted digital life in general. Cyber threats include, but are not limited to, malware, spyware, phishing attacks, ransomware, zero-day exploits, trojans, advanced persistent threats, wiper attacks, data manipulation, data destruction, rogue software, malvertising, unpatched software, computer viruses, man-in-the-middle attacks, data breaches, Denial of Service (DOS) attacks, and other attack vectors.

The term “hash value” used herein can be thought of as fingerprints for files. The contents of a file are processed through a cryptographic algorithm; and a unique numerical value, the hash value, is produced that identifies the contents of the file. If the contents are modified in any way, the value of the hash will also change significantly. Example algorithms used to produce hash values are the Message Digest-5 (MD5) algorithm and Secure Hash Algorithm-1 (SHA1).

The term “integrity check” as used herein refers to the checking for accuracy and consistency of system related files, data, etc. It may be performed using checking tools that can detect whether any critical system files have been changed, thus enabling the system administrator to look for unauthorized alteration of the system. For example, data integrity corresponds to the quality of data in the databases and to the level by which users examine data quality, integrity, and reliability. Data integrity checks verify that the data in the database is accurate, and functions as expected within a given application.

The term “alarm” as used herein refers to a trigger when a component in a system or the system fails or does not perform as expected. The system may enter an alarm state when a certain event occurs. An alarm indication signal is a visual signal to indicate the alarm state. For example, when a cyber security threat is detected, a system administrator may be alerted via sound alarm, a message, a glowing LED, a pop-up window, etc. An alarm indication signal may be reported downstream from a detecting device to prevent adverse situations or cascading effects.

The term “in communication with”, as used herein, refers to any coupling, connection, or interaction using electrical signals to exchange information or data, using any system, hardware, software, protocol, or format, regardless of whether the exchange occurs wirelessly or over a wired connection.

As used herein, the term “cryptographic protocol” is also known as security protocol or encryption protocol. It is an abstract or concrete protocol that performs a security-related function and applies cryptographic methods often as sequences of cryptographic primitives. A protocol describes how the algorithms should be used. A sufficiently detailed protocol includes details about data structures and representations, at which point it can be used to implement multiple, interoperable versions of a program. Cryptographic protocols are widely used for secure application-level data transport. A cryptographic protocol usually incorporates at least some of these aspects: key agreement or establishment, entity authentication, symmetric encryption, message authentication material construction, secured application-level data transport, non-repudiation methods, secret sharing methods, and secure multi-party computation. Hashing algorithms may be used to verify the integrity of data. Secure Socket Layer (SSL) and Transport Layer Security (TLS), the successor to SSL, are cryptographic protocols that may be used by networking switches to secure data communications over a network.

As used herein, the term “network” may include the Internet, a local area network, a wide area network, or combinations thereof. The network may include one or more networks or communication systems, such as the Internet, the telephone system, satellite networks, cable television networks, and various other private and public networks. In addition, the connections may include wired connections (such as wires, cables, fiber optic lines, etc.), wireless connections, or combinations thereof. Furthermore, although not shown, other computers, systems, devices, and networks may also be connected to the network. Network refers to any set of devices or subsystems connected by links joining (directly or indirectly) a set of terminal nodes sharing resources located on or provided by network nodes. The computers use common communication protocols over digital interconnections to communicate with each other. For example, subsystems may comprise the cloud. Cloud refers to servers that are accessed over the Internet, and the software and databases that run on those servers.

The present disclosure provides devices, methods, and systems to assess the status of a bowel preparation in a subject. In an embodiment, the device, the methods, and the system assess the status of bowel preparation in a subject in real-time. technology is built on four key pillars: a) an auto image capturing device that can acquires real-time images with appropriate backgrounds for colon readiness analysis, b) an image analysis unit that is configured to measures parameters (such as turbidity, color, particulate concentration, size distribution) to assess bowel preparation quality, c) a sampling method and biochemical and chemical analysis procedure that allows additional analysis of rectal effluent for multiple health related markers and indicators, and d) AI Integration that provides actionable insights for better patient outcomes. The impact of this technology is multifold. It increases the success rate of colonoscopies by determining if additional bowel cleansing is necessary six hours before the procedure. If a patient requires more laxatives, it can be administered in advance to ensure a clean colon. Alternatively, if the bowel remains unclean, the appointment can be rescheduled. This leads to cost savings for insurance companies by reducing repeat procedures and hospitalizations. It also improves patient experience by ensuring successful colonoscopies and leads to better resource utilization by reducing missed lesions and adverse events.

An embodiment relates to a method to determine the status of colon cleansing in subjects prior to a gastrointestinal procedure. The method comprises providing a reference background for measuring plurality of parameters, capturing an image of the rectal effluent; assessing the image for readiness of bowel preparation, measuring the plurality of parameters for assessing the image for readiness of bowel preparation, and providing an assessment of bowel preparation to undergo colonoscopy.

In an embodiment, the method utilizes a high-resolution image capturing that allows even minute traces of a target substance to be detected if even only a single pixel in an image corresponding to the target substance is captured. The method can be used to detect any kind of solid substance that has spectral characteristics that can be viewed within at least portions of the fields of view of the image capture array. Individual pixels, or groups of pixels, of captured image information can be analyzed to assess if the pixel(s) display spectral characteristics of the target substance.

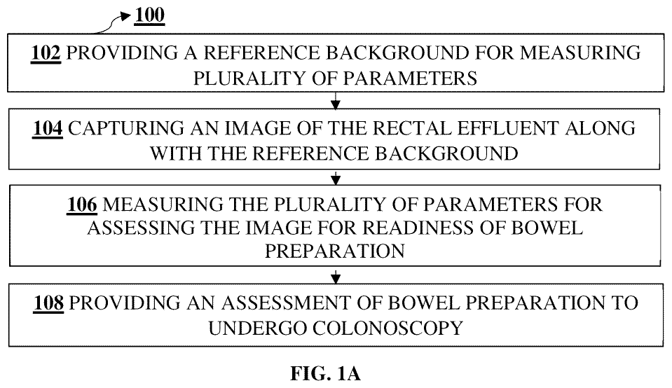

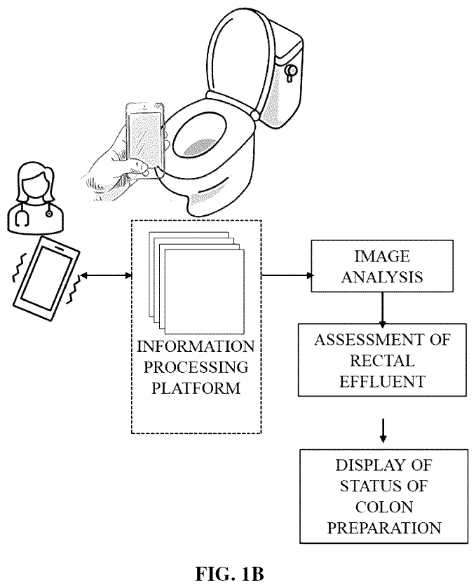

Referring to FIG. 1A, it illustrates a method for assessing readiness of colon preparation, according to an embodiment. At 102, method 100 comprises providing a reference background for measuring plurality of parameters. At 104, the method 100 comprises capturing an image of the rectal effluent along with the reference background. At 106, the method 100 comprises measuring the plurality of parameters for assessing the image for readiness of bowel preparation. At 108, the method 100 comprises providing an assessment of bowel preparation to undergo colonoscopy.

In another embodiment, the method utilizes an app comprising a reference background to ensure consistency and accuracy in the captured images

Referring to FIG. 1B, it provides a pictorial presentation of method for assessing readiness of colon preparation, according to one or more embodiments. The process involves the following steps: The user installs a dedicated application on their mobile device. This application is designed to facilitate the capture and analysis of images related to colon preparation. The user utilizes the app to capture an image of the rectal effluent. The app comprises a reference background to ensure consistency and accuracy in the captured images. During the image capture process, the app provides real-time feedback and indications to help the user adjust the image for optimal quality. This may include suggestions for lighting, angle, and focus. Once the image is captured, the app evaluates its acceptability for analysis. If the image does not meet the required standards, the app prompts the user to retake the image with specific adjustments. After capturing an acceptable image, it is transmitted to an information analysis unit. This unit employs advanced algorithms to analyze the image and assess the quality of the rectal effluent. The processed image is analyzed to determine the readiness of the colon preparation. The results of this analysis are then displayed on the user's device, providing a clear status update on the colon preparation. This detailed approach ensures that the user is guided through each step, from app installation to receiving the final assessment, enhancing both accuracy and user experience.

The phone app may instruct the subject not to flush after excreting, as well as not to place toilet paper in the sample funnel or sample container. Additionally, the phone app may guide the subject by using the app's camera function to take a top-down photo of the sample collection device. A phone image is taken directly from the top, looking down on the analysis cell. The phone app assists in taking a proper image and sending it to the server for analysis. A sample image may also be provided, and the app can assist the subject in confirming if the captured image is of the correct size by displaying an outline of the sample collection container. The app may further adjust contrast, brightness, focus, and other parameters as necessary to obtain the best images. Moreover, a tab on the collection device can be pulled up to release the adhesive and flush the collection device down the toilet or dispose of it in the trash. The app may also indicate the subject when a correct image is captured. Further, a tab on the collection device may be pulled up to release the adhesive and flush the collection device down the toilet or dispose of it in the trash. The app may also take care of sending the captured images and the identification number of the device to a server for processing. In some embodiments, the app can be removed from the phone after use.

The user logs into a dedicated cell phone app and inputs the patient's information. This app provides a secure and reliable platform for viewing and storing captured images. After necessary adjustments, the framed device is placed inside the toilet. The device features adjustable support bars between the camera and the target. These bars help stabilize the device, ensuring steady and clear image capture. A test image is taken to make sure one of the focus planes of the target is acceptable. This feature ensures that the images captured are sharp and clear, providing valuable insights into the patient's condition. The device is designed to take an image before every flushing. The time of the image is recorded by the app. The user continues to use the device as instructed by the app. The captured images are submitted through the app. The app also allows for easy sharing of the images with healthcare professionals.

The images can be viewed by the doctor through the app or analyzed by the built-in AI in the app. The images are either approved by the doctor or automatically by the app with the built-in AI.

An embodiment relates to a device comprising: a disposable assembly comprising a hook, an adjustable frame, and an endoscope; and a reusable assembly comprising a detachable USB cable configured to connect with an external computing device; wherein the device is configured to capture images of rectal effluent.

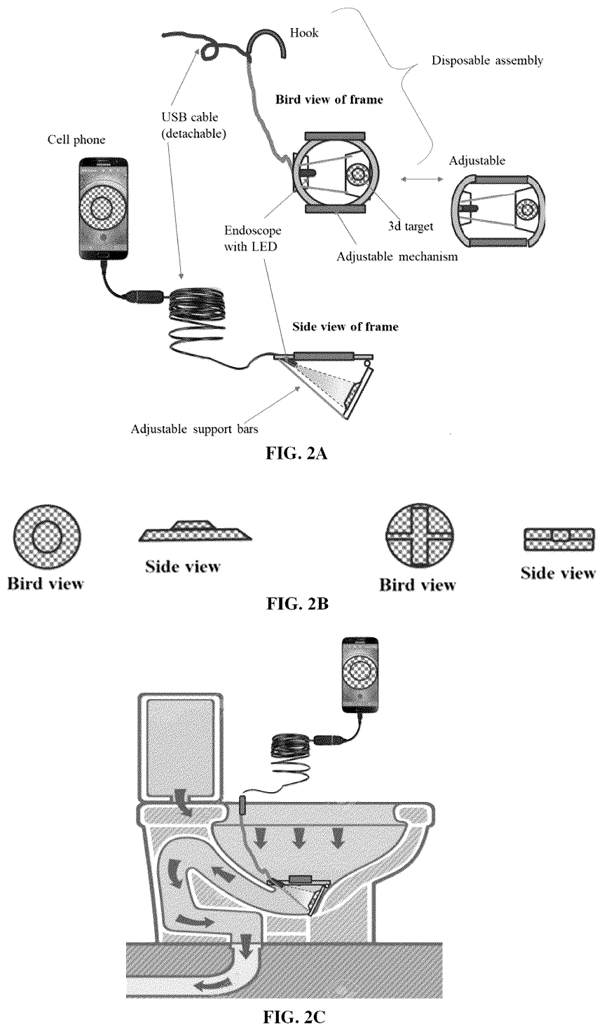

Referring to FIG. 2A, the device comprises a disposable assembly designed for single use to maintain hygiene and prevent cross-contamination, and a reusable assembly comprising components that can be used multiple times. The disposable assembly comprises a hook, an adjustable frame, and an endoscope. The hook secures the device to the toilet seat, ensuring stability during use.

The adjustable frame can be adjusted via a mechanism to fit different toilet designs, allowing the device to be used in various settings. In some embodiments, the endoscope is equipped with an LED light source to capture clear images of the rectal effluent, ensuring adequate illumination for high-quality images even in low-light conditions.

The reusable assembly comprises a USB cable, which includes a connector and a port. This detachable USB cable is used for power supply and data transfer, allowing for easy replacement in case of damage or wear.

The device features adjustable support bars between the camera and the target in some embodiments. These bars help stabilize the device, ensuring steady and clear image capture. Additionally, the device uses a 3D target with different focus planes to facilitate easy focusing, ensuring that the images captured are sharp and clear, providing valuable insights into the patient's condition.

Referring to FIG. 2B, it shows example views of the target, according to one or more embodiments.

Referring to FIG. 2C, it shows a pictorial representation attaching the device to a commode, according to one or more embodiments.

In some embodiments, a device is provided, the device comprising a flat rod comprising a first end and a second end, the first end to configured act as a hook for stability, and the second end comprising a camera for image acquisition. The flat rod is sturdy, durable, and of lightweight, made up of alloy or plastic sturdy rod. The hook allows user to position the device securely within the commode. The second end comprises a miniature camera module and an LED illumination for optimal imaging. It captures high-resolution images of rectal effluent. The second end is submerged in the effluent during use.

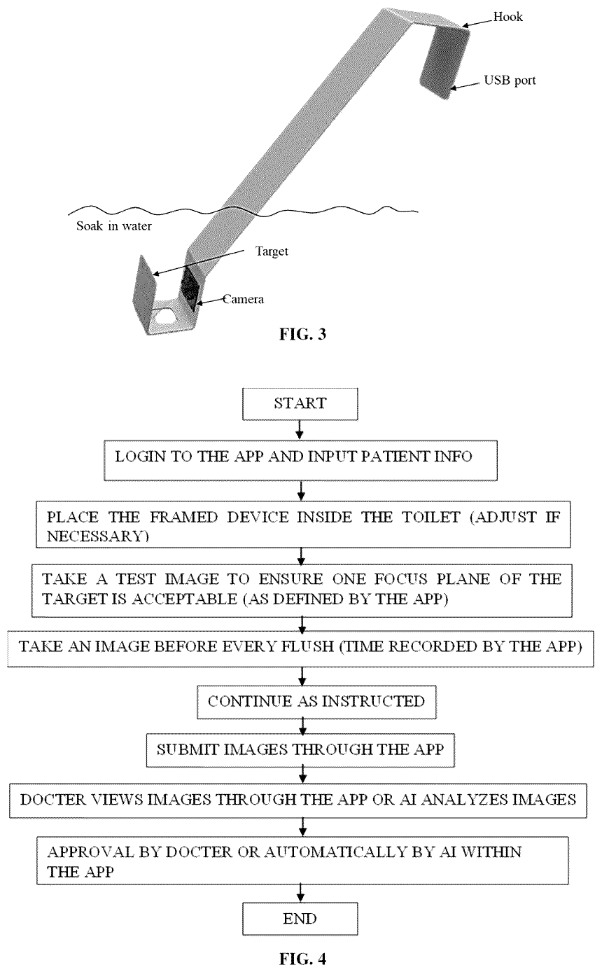

Referring to FIG. 3, the device features a flat rod made from a lightweight and durable alloy or plastic is designed to be straight with a slight curve at the hook end, providing both stability and ease of use during medical examinations. The material choice ensures that the rod is both robust and manageable, making it ideal for repeated use in clinical settings. The hook end of the device is configured into a rounded shape, enabling secure positioning within a commode. This design ensures that the device remains stable during use, preventing any displacement that could affect the quality of the imaging. The hook's curvature is carefully crafted to balance ease of placement with secure attachment. The imaging end of the device is equipped with a miniature camera module and LED illumination. These components work together to capture high-resolution images of the rectal effluent. The camera module provides clear visualization, while the LED lights ensure optimal imaging conditions, even in less-than-ideal lighting environments. The imaging end is designed to be submerged in the effluent, allowing for detailed capture of its characteristics.

Before using the device, it is essential to ensure proper hygiene and sterilization to maintain patient safety and device integrity. The hook end is positioned securely within the commode to provide a stable base for the imaging process. Once the device is in place, the imaging end is dipped into the rectal effluent. The camera module is then activated, and the angle adjusted for the best possible visualization. The user can capture images as needed, depending on the specific diagnostic requirements. Captured images are stored on an external device, such as an SD card, allowing for easy transfer and analysis. These images can be transferred to a computer for further examination and documentation, facilitating detailed analysis by healthcare professionals.

The device can be connected to an external computing device such as a is enabling the user to view the captured images in real-time and store them for future reference.

In some embodiments, the device comes with a dedicated mobile app that provides a secure and reliable platform for viewing and storing the captured images. The app also facilitates easy sharing of the images with healthcare professionals.

Referring to FIG. 4, it illustrates a flowchart for using the device described in the various embodiments. After logging into the dedicated mobile app and inputting the patient's information, the user places the adjusted device inside the toilet. The device is designed to take an image before every flush, with the time of the image recorded by the app. The user continues to use the device as instructed by the app, and the captured images are submitted through the app.

The images can be viewed by the doctor through the app or analyzed by the built-in AI in the app. The images are either approved by the doctor or automatically by the app with the built-in AI.

In some embodiments, the device comprises a Wi-Fi module, allowing it to connect wirelessly to the internet for seamless data transfer.

In some embodiments, the device may also comprise a battery indicator on the reusable assembly, providing real-time information about the device's power status.

In some embodiments, the device comprises a waterproof casing for the electronic components, ensuring its durability and longevity even in moist conditions.

In some embodiments, the dedicated mobile app can send reminders to the user to charge the device or replace the disposable assembly, enhancing the user experience.

In some embodiments, the app comprises a data encryption feature, ensuring the privacy and security of the user's medical data.

The built-in AI in the app can provide preliminary analysis of the images, highlighting areas of concern and providing suggestions for further medical consultation.

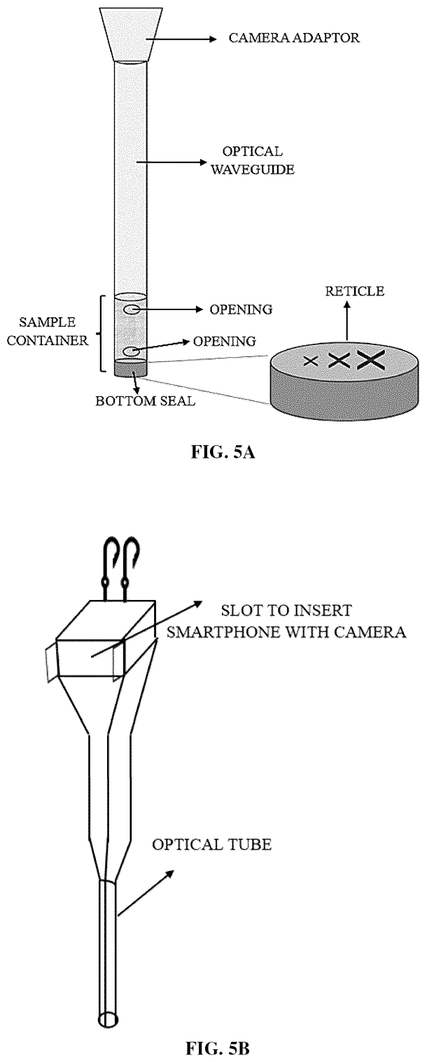

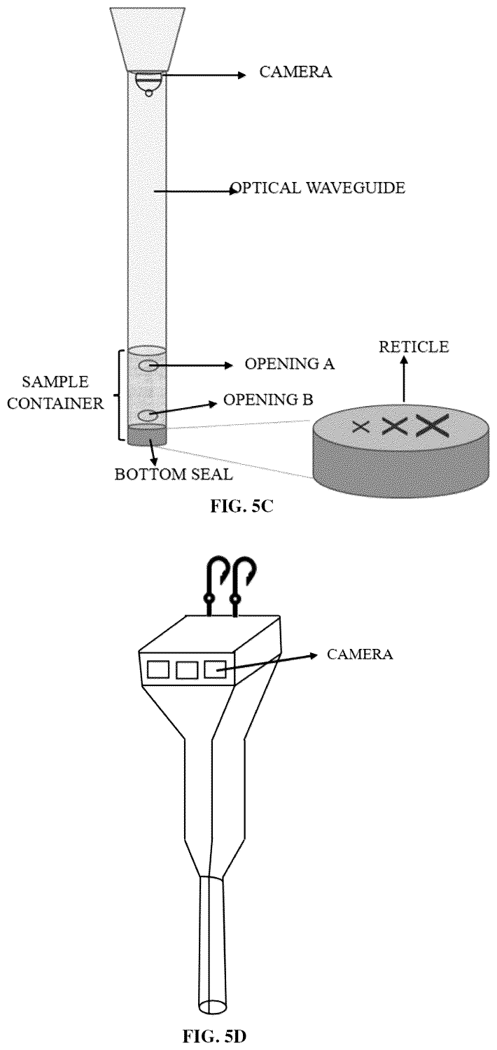

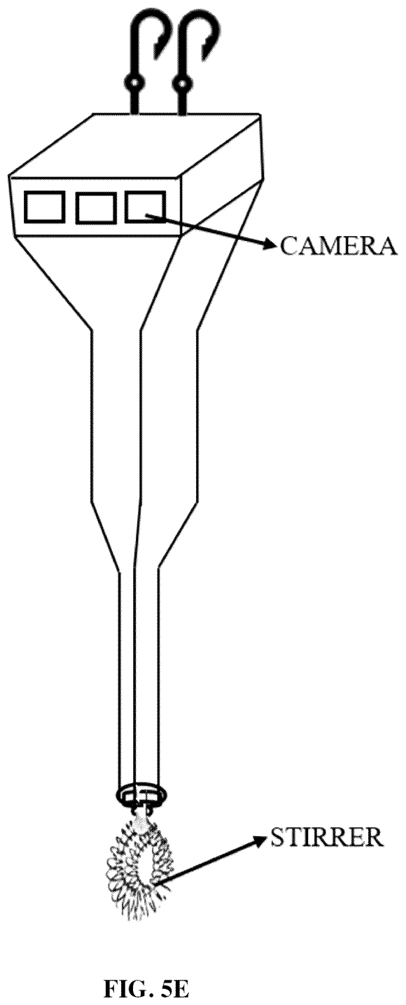

Referring to FIG. 5A, the device comprises an adapter that connects to a smartphone. A flexible long optical waveguide guides directs light from the adapter to the toilet bowl. The flexible long optical waveguide can be made of plastic, fiber, or glass and may be a single piece or expandable. The device comprises a hollow space within the device that acts as a sample container. The sample container serves as a space for rectal effluent accumulation. One or more openings facilitate liquid entry into the cavity. The one or more openings allow liquid to flow in and out into the sample container. To prevent flocking, the liquid needs to circulate and flush the toilet multiple times. A reticle at one end of the cavity helps the camera record color and cleanliness information. The camera captures images, which can be sharp or cloudy depending on the liquid's state (e.g., yellow color indicates cleanliness).

In an embodiment, the device allows the users to place their camera inside the device. An external device synchronizes with the camera via Bluetooth. When the user presses a button on the external device, the message is wirelessly transmitted to the camera to capture pictures.

In an embodiment, the adaptor of the device can hold a smart phone and camera of the smart phone is used to capture the image. Referring to FIG. 5B, it shows a device comprising an adaptor to hold a smart phone.

In some embodiments, a top part of the device comprises a built-in camera. This component is fully disposable and can be replaced after use. FIG. 5C and FIG. 5D show devices comprising built-in camera, according to one or more embodiments.

In some embodiments, after the user completes the test, the device collects prep samples. These samples may be used to provide full-body intelligence about the gut biome. Users can seal the disposable component, place it in a pouch, and mail it for analysis.

In some embodiments, by combining colonoscopy readiness assessment with gut biome analysis, the device ensures comprehensive health insights. The two components (colonoscopy readiness and gut biome) are integrated, replacing the need for separate tubes.

An embodiment relates to a device comprising: an imaging portion configured to capture an image of the rectal effluent; a stirrer configured to homogenize a rectal effluent; and a communication unit configured to transmit the image to a user system.

In an embodiment, the stirrer is connected to a motor unit. In another embodiment, the motor unit comprises an electrical drive motor. In yet another embodiment, the electrical drive motor is coupled to and arranged for driving a motor drive shaft of the motor unit. In yet another embodiment, the electrical drive motor is powered by a battery. In yet another embodiment, the electrical drive motor is powered by a main electrical line. In yet another embodiment, a rotation speed of the stirrer is adjustable via the motor unit. In yet another embodiment, the stirrer is removably connected to the device via a port. In yet another embodiment, the stirrer comprises sensors to detect frothing. In yet another embodiment, the device is configured to generate an alert if frothing occurs beyond a set threshold. In some embodiments, the speed of the stirrer is adjustable. In another embodiment, the speed is adjusted manually adjusted using a knob or a dial on the stirrer. In yet another embodiment, the speed is adjusted using a programmable logic controller. FIG. 5E illustrates a device comprising stirrer, according to one or more embodiments.

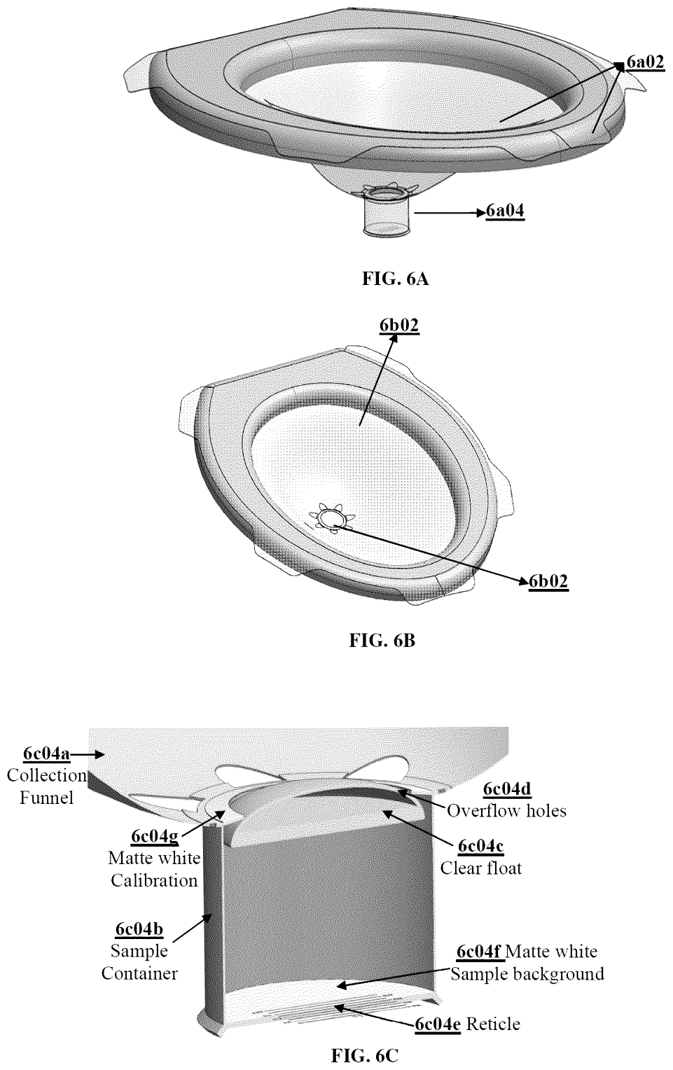

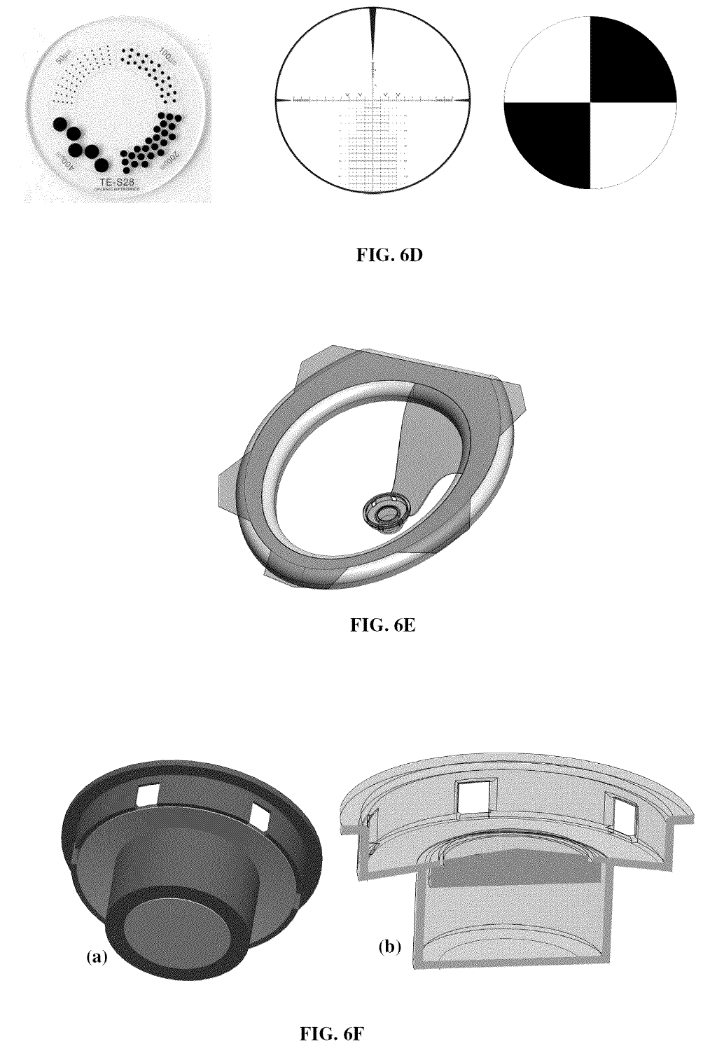

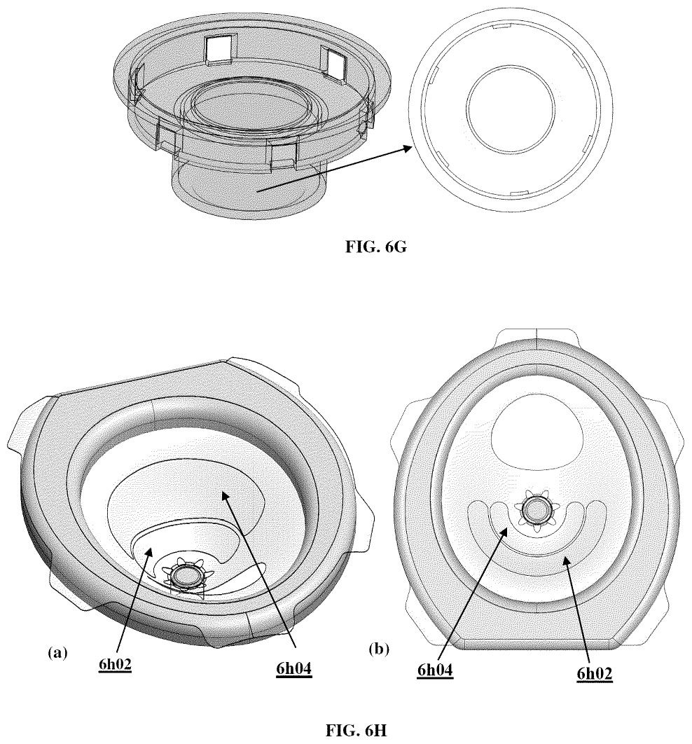

Referring to FIG. 6A, it illustrates a flushable device designed to be attached to a toilet commode, according to one or more embodiments. The device comprises a sample funnel 6a02 and a sample container 6a04. The sample funnel 6a02 is configured to channel the rectal effluent to the sample container 6a04.

In an embodiment the sample funnel 6a02 comprises a bungee type means of attachment. In another embodiment, the sample funnel 6a02 comprises an adhesive type means of attachment. The sample funnel may comprise an adhesive on one side using which the device can be stuck to a commode seat. The adhesive can be a waterproof adhesive to seal flushable materials. The adhesive can be one of a water-based emulsion adhesives, an acrylic rubber-based adhesive, and a cross-link coating.

Referring to FIG. 6B, it illustrates (i) bungee type and (ii) adhesive type of device, according to one or more embodiments. In some embodiments, the sample funnel 6b02 is made up of a flushable material. In some embodiments, sample funnel 6b02 comprises cellulose fibers and a water insoluble film to increase the tensile strength of the flushable material. In yet another embodiment, the sample funnel 6b02 is made up of a biodegradable material. In some embodiments, the sample funnel 6b02 configured with varying levels of thickness to conserve materials and increase the strength of the seat cover at different locations. In some embodiments, the sample funnel 6b02 uses curtain coating or another method to create the bilayer film. In some embodiments, the sample funnel 6b02 comprises a waterproof adhesive or cross linking to seal/combine two or more pieces.

In some embodiments, the sample funnel 6b02 can be made up of a strong material. The material is strong enough to hold an analysis cell weight and a possible dropping of a mobile phone. In some embodiments, the sample funnel 6b02 can be attached to a commode seat by releasing pull tabs of the device on the periphery. In some embodiments the sample funnel 602 comprises printed instructional images.

Referring to FIG. 6C, it illustrates a transparent view of sample container of the flushable device, according to one or more embodiments. The matte white calibration surface 6c04g provides a calibration white for the camera while the matte white sample background 6c04f provides a bright white background to see the bowel fluid color and particulates in the fluid. A reticle 6c04e is printed on the bright white circle to help with particulate count and size.

The sample container 6c04b comprises an analysis cell. The rectal effluent can flow down the sample funnel to fill the sample container 6c04b. Referring FIG. 6C, at the cross-section image of the analysis cell, the water-soluble funnel attaches to the sample container beneath it. The sample container may further comprise a float 6c04c. The float 6c04c is an internal float. It is water clear. This allows a direct downward view of the reticle 6c04e on the bright white surface. It can be provided to a subject as a flat package. The float 6c04c is initially at the bottom of the sample container. As the rectal effluent flows down the collection funnel 6c04a, it fills up the analysis cell 6c04b. The float 6c04c rises on top of the fluid eventually reaching the top where it prevents further liquid flow (and more importantly, additional solids) from entering the analysis cell 6c04b. At this point, all excess rectal effluent flows out of the overflow holes 6c04d at the bottom of the funnel and into the toilet bowl.

In an embodiment the sample container comprises a single cell fill, a calibration section, and an image background. The single cell fill can fill only once, and a fluid should not cycle through, or particulates may settle during fill. The single cell fill comprises an optically clear cylinder float. In some embodiments, the sample container is made up of cellulose material. The calibration section allows for uncalibrated user phones. A disk is split between sample container and calibration section. The disk may be center split or calibration or calibration surrounding round sample section (ring). The image background is flat bottom for even settling. It can be bright white with a matte finish for reduced glare spots and therefore there will be no camera dependence.

FIG. 6D illustrates reticle options that can be used in the device, according to one or more embodiments.

In one embodiment, the device is a flushable device that can be taken home. It may require minimal setup by the subject with low complexity. In some embodiments, the device is clean, hazmat free and disposable.

At a recommended time, the subject can open the flat packaged device and following a visual phone app-based instruction can expand the sample funnel and the sample container. Next, the subject should remove the adhesive backing from a first side of the device and adhere the device on a toilet seat. A reference photo setting may be configured to confirm that the device is properly placed on the toilet seat.

During the next bowel movement, the subject may use the toilet as usual, but may avoid urinating into the sample funnel, and not flush after excreting. In an embodiment, a first portion of the funnel is open and is configured to allow most of the urine to bypass entering the sample container. In another embodiment, a slanting wall may be added to a second portion near to the sample container to only allow rectal effluent to enter the analysis cell.

In an embodiment, the device comprises an identification number.

The phone app may instruct the subject not to flush after excreting, as well as not to place toilet paper in the sample funnel or sample container. Additionally, the phone app may guide the subject by using the app's camera function to take a top-down photo of the sample collection device. A phone image is taken directly from the top, looking down on the analysis cell. The phone app assists in taking a proper image and sending it to the server for analysis. A sample image may also be provided, and the app can assist the subject in confirming if the captured image is of the correct size by displaying an outline of the sample collection container. The app may further adjust contrast, brightness, focus, and other parameters as necessary to obtain the best images. Moreover, a tab on the collection device can be pulled up to release the adhesive and flush the collection device down the toilet or dispose of it in the trash. The app may also indicate the subject when a correct image is captured. Further, a tab on the collection device may be pulled up to release the adhesive and flush the collection device down the toilet or dispose of it in the trash. The app may also take care of sending the captured images and the identification number of the device to a server for processing. In some embodiments, the app can be removed from the phone after use.

In some embodiments, the image is processed at a remote server to color correct the image (white ring as calibration), detect the color of the bottom circle, count particulates and determine size distribution, etc. The results can be compared to the limits set by the healthcare provider to determine the patient's readiness for the procedure. Other methods or algorithms may be used to determine patient's readiness.

In an embodiment, a single cell fill mechanism is designed with specific considerations to ensure precise and efficient cell filling while maintaining optimal imaging conditions. The cell can only be filled once to prevent any potential issues that may arise from cycling through the process, ensuring accurate results. To aid in this process, an optically clear cylinder float is utilized, allowing for clear visibility during cell filling.

FIG. 6E illustrates a partial view of disposable seat cover, according to one or more embodiments.

FIG. 6F illustrates a bottom view and a cross-sectional view of the analysis cell from different angles.

FIG. 6G illustrates an analysis cell comprising a reticle, according to one or more embodiments.

The calibration section is a critical component that accommodates uncalibrated user phones, making the device more user-friendly. The disk is divided between the sample container and the calibration section, and options are available for a center split or a calibration surrounding the round sample section (ring), depending on the required depth of the disk.

In an embodiment, to optimize imaging quality, the image background is designed with a flat bottom to facilitate even settling. It is bright white with a matte finish, reducing glare spots and eliminating camera angle dependence, ensuring consistent and reliable imaging. A reticle on the background assists in measurements and precise analyses.

Additionally, in one or more embodiments, the device is disposable for convenience and hygiene. If possible, cellulose construction is employed, and if not, gloves and a pull handle are provided for easy handling. To prioritize sustainability, cellulose/PLA materials are used for the cell, making it biodegradable. Furthermore, a gel-based float inside the bag is flushable and biodegradable, adding to the eco-friendly features of the device. Overall, the Single Cell fill mechanism is meticulously designed to deliver accurate and environmentally conscious results for various medical and research applications. In an embodiment, the analysis cell may be read in the toilet or may be removed for reading.

In one embodiment, the device comprises a triple sensor system that facilitates color and turbidity measurement. The device is pre-calibrated upon package opening, streamlining the setup process. One distinctive feature is its ability to operate both in and out of the toilet without requiring any motion from the user. The results are communicated to the client's phone via pulsed infrared technology, offering quick and efficient data transfer. The device can be powered by a watch battery and a cost-effective microcontroller, ensuring long-lasting and economical usage.

In one embodiment, the device comprises a paddlewheel. This embodiment takes advantage of a paddlewheel or similar rotary motion mechanism to achieve comprehensive measurements, including color, turbidity, particle size, and distribution accuracy. It employs either a line pattern or spot sensor, allowing it to function efficiently both in and out of the toilet. The client is actively involved in providing swirling motion during use, enhancing the accuracy of the results. A stick is provided for in-toilet usage, and the outer edge of the paddlewheel offers calibration capabilities. The inner hub features a matte white finish for optimal imaging conditions, while the rotational speed, along with known volume, is utilized to calibrate particulate size and count. Additionally, this embodiment can be used with a separate reader if required.

In an embodiment, the device is an out of toilet reader device. The said device caters to scenarios where a return unit to the office for a new disposable cover is feasible. It features a magnetic stirrer integrated into the paddlewheel for efficient mixing during analysis. The device is designed for constant flow, adopting a cell counter style. Optics are employed to determine particulate size by measuring light blockage, magnifying the tube contents for accurate results.

In an embodiment, the device comprises a stickiness indicator that is configured to aid in measuring stickiness of bowel fluid. The stickiness indicator may comprise one or more of the following:
a) A surface on the collection funnel: It may be positioned at or below in the device where the bowel fluid stream initially comes in contact with the collection funnel. The material of this surface is designed to promote the attachment of the sticky material. Additionally, a chemical treatment may be applied to bond chemically with the sticky material. The surface may also be coated with a mesh, further encouraging the adherence of sticky material. A chemical may be added to the surface to enhance visualization, and its color may be optimized for better visualization.
b) A post within the sample container: It may be positioned such that the post comes into contact with the bowel fluid entering the container. Its surface material is designed to facilitate the attachment of the sticky material. Similar to the surface, the post may also be coated with a mesh and treated with chemicals to promote bonding with the sticky material. Visualization of the sticky substance can be improved by adding a chemical or using a specific color.
c) A mesh or high surface area material covering the sample container opening: It may be positioned to come in contact with the bowel fluid entering the container. The mesh is designed to foster attachment of the sticky material and may be chemically treated for better bonding. Visualization can be enhanced by adding a chemical or using a specific color.
d) Two close, opposing surfaces: It may be placed within the flow of bowel fluid, allowing it to fill the gap between them. After the bowel movement, the force required to separate the surfaces can be measured electronically or visually using a gauge or similar method.
e) Other industry-standard stickiness measurement methods: Any other method commonly used in the industry to measure stickiness can also be incorporated into the device.

Electronic images of the stickiness indicator will be utilized to determine the stickiness or relative stickiness of the bowel fluid. Electronic or electric sensors may be attached to the stickiness indicator to provide a relative measurement of stickiness using capacitance, resistance, or other electrical values. The stickiness indicator materials may be designed to be flushable for convenience and ease of use.

In an embodiment, the device comprises a viscosity indicator that is configured to aid in measuring viscosity of bowel fluid. In some embodiments, the viscosity indicator material is designed to be flushable, ensuring easy disposal.

The viscosity indicator may comprise one or more of the following:
a) Maze-like surface on the collection funnel: Positioned below where the bowel fluid stream initially contacts the collection funnel, designed to slow down the movement of viscous bowel fluid as it slides down the funnel's sides. The surface may have ridges, bumps, or other obstacles, as well as a rough texture or chemical treatment to further impede the fluid's movement. A mesh version of this surface is also considered. A chemical may be added for better visualization, and the surface color may be optimized accordingly.
b) Flow through an orifice: A portion of the bowel fluid is forced to flow through an orifice, and the flow rate is measured visually or mechanically to determine viscosity.
c) Fall rate of an object through the fluid: A ball, weighted mesh, or another object is released through the bowel fluid, and its fall rate is measured visually or mechanically to calculate viscosity. Obstacles or a track can be used to extend the measurement time.
d) Consistometer method: Measuring the speed at which bowel fluid moves down a track as a method of determining viscosity.
e) Other industry-standard viscosity measurement methods: Various other methods used in the industry to measure dynamic or kinematic viscosity, such as capillary viscosity, rotational or vibrating viscometers, and microfluidic rheometers, may be included.

FIG. 6H show a stickiness indicator and a viscosity indicator of the device, according to one or more embodiments. In some embodiments, the stickiness indicator 6h04 and viscosity indicator 6h04 of the device may share the same mesh component. Additionally, an alternate embodiment of the sample container may comprise an extension of the funnel rather than a separate container, while the mesh may be used for particulate counting.

In an embodiment, the device is a user-friendly cylindrical device.

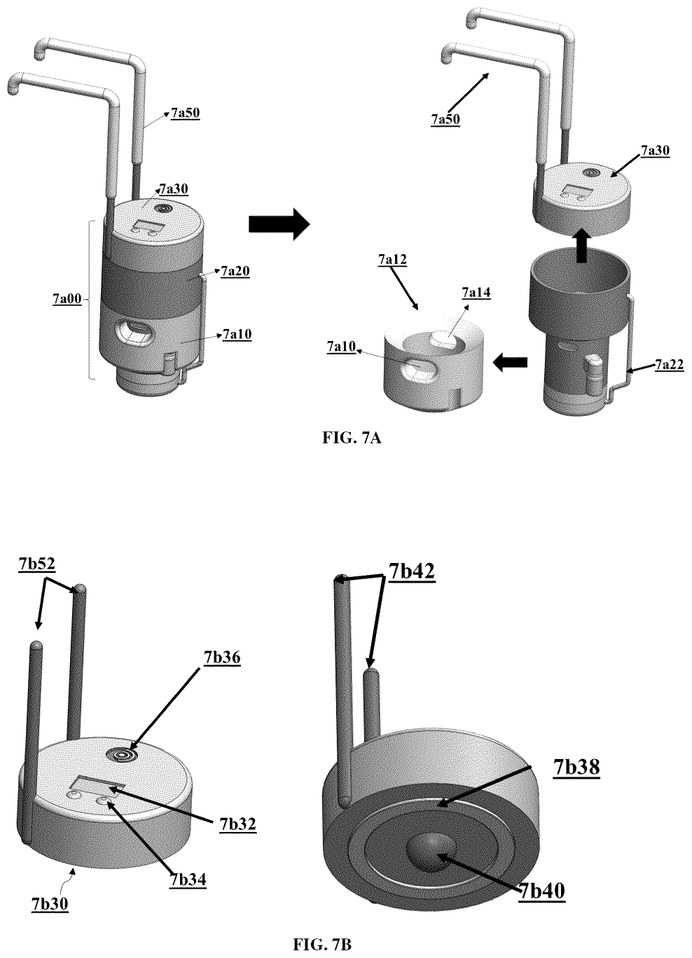

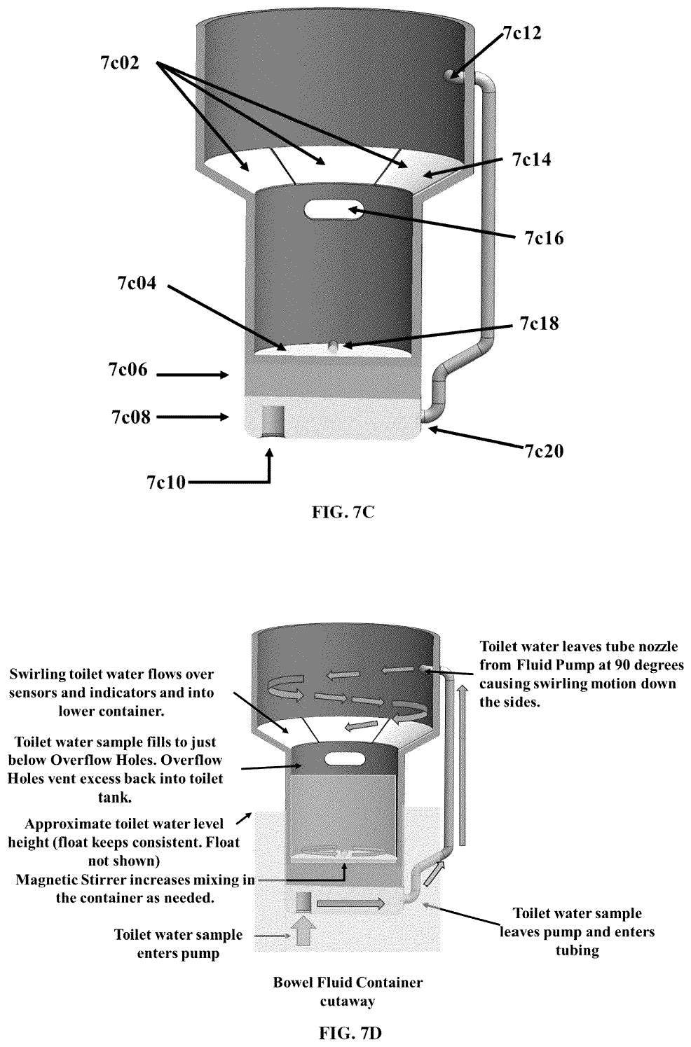

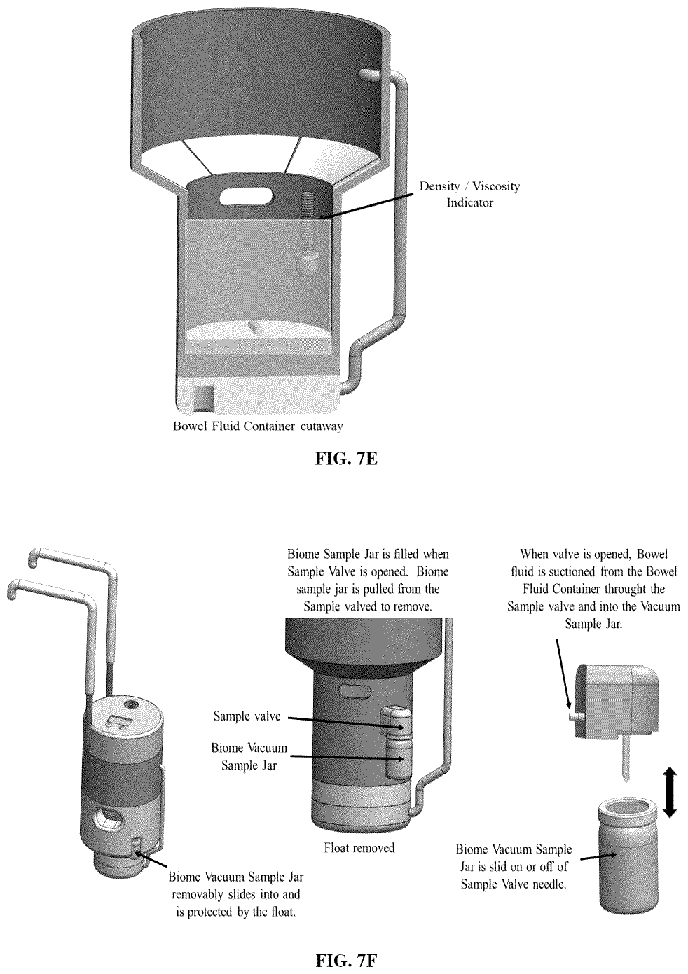

Referring to FIG. 7A, it illustrates a colonoscopy readiness device, according to one or more embodiments. The device comprises a cylindrical body 7a00, a float 7a10, a fluid container 7a20, an electronics unit 7a30, and an attachment unit 7a40. The float 7a10 comprises an inlet 7a12 and an outlet 7a14. The electronics unit 7a02 comprises a camera, a lighting unit, a communication component, and a display. In some embodiments, the fluid container 7a20 comprises one or more sensors (not shown in the figure) and a sampling unit 7a22. In some embodiments, the attachment unit is length adjustable.

Referring to FIG. 7B, it illustrates a top view and a bottom view of the electronics unit of the colonoscopy readiness device, according to one or more embodiments. The electronics unit 7b30 comprises one or more of an output component 3b32, an input component 3b34, and optionally an auditory input/output component 3b36. Further, the face of the electronic unit 3b30 towards the fluid container 7b04 of FIG. 7A comprises a light Source 7b38 and a camera 7b40. The light source 7b38 will be used to illuminate the fluid container and the camera comprising lens will be used to capture images and videos of the fluid container.

In an embodiment, the fluid container comprises a multi-speed fluid pump, a fluid pump inlet, a fluid pump outlet, and a tube nozzle. In some embodiments, the fluid container further comprises a pump motor and a magnetic stirrer. In some embodiments, the device further comprises matte white background and reticle. The device further comprises a viscosity indicator, a stickiness indicator, and a color and gray scale calibration image. The camera of the electronics unit looks down on sensors, viscosity indicators, stickiness indicators, Color and gray scale calibration images and captures audios and videos of the toilet water and various other measurable parameters listed somewhere else in this specification. The matte white background and reticle enhances camera ability to determine fluid color, turbidity and detect particulates. The tube nozzle directs fluid parallel to walls causing swirling motion down the sides. The swirling toilet water flows over sensors and indicators and into a lower container. The magnetic stirrer increases mixing through swirling the fluid. The fluid pump inlet pumps fluid from toilet tank water and the fluid pump outlet pumps the toilet fluid into tubing. The multi-speed fluid pump helps increase or decrease pumping speed based on the pumping requirements.

The one or more sensors comprise a chemical sensor, a visual sensor, an electric sensor, and an electronic sensor. In some embodiments, the sensors comprise a replaceable, disposable sensor covers to ensure hygiene and accuracy. In some embodiments, the sensors themselves may be replaceable for convenient maintenance. In some embodiments, the device may comprise a display or LEDs to provide patients with clear and accessible result information.

In some embodiments, the device comprises a wireless link to a server to enable seamless data processing and communication. A wireless link can facilitate the transmission of images and other sensor data to the server for processing. It can also enable the transmission of commands, such as configuration, testing, and monitoring instructions, from the server to the device. The wireless link may utilize various technologies, comprising cellular, Wi-Fi, and Bluetooth connections.

In some embodiments, the device comprises a mixer to aid in fluid flow control into the camera's field of view. In some embodiments, the device comprises a force-means to force the flow of fluid into the camera's field of view. The non limiting examples of force-means are a pump and a mixer.

In some embodiments, the device comprises an estimation means to estimate or measure the initial toilet water volume. This measurement or estimation will allow calculation of the particle count per volume. In some embodiments, the device comprises sound or vibration sensors. These sensors can serve various purposes, such as detecting when the user is seated, experiencing a bowel movement, getting off the seat, or when the toilet has been flushed. In some embodiments, the device comprises a user input feature (for example a button). The user input feature will enable the patient to initiate the device's measurements conveniently. In some embodiments, the device comprises an indicator to convey the device's status to the user or to signal whether the toilet should or should not be flushed. The indicator can be one of more of a light indicator, a sound indicator, and a voice indicator.

In some embodiments, the device is configured to provide clear instructions, delivered through verbal, visual (e.g., display or lights), or auditory means, to guide the user throughout the process. Additionally, in case of any errors, the device can deliver user-friendly instructions and include relevant contact information.

In some embodiments, the device will incorporate a test function for the toilet to determine baseline values of the current sensor values. This functionality is especially critical due to potential variations in water quality, ensuring accurate and reliable performance.

Referring to FIG. 7C, it illustrates a cutaway of the fluid container of the device, according to one or more embodiments. The Camera fluid container comprises a multi-speed fluid pump, a fluid pump inlet, a fluid pump outlet, and a tube nozzle. In some embodiments, the fluid container further comprises a pump motor and a magnetic stirrer. In some embodiments, the device further comprises matte white background and reticle. In some embodiments, the bowel fluid container comprises an overflow hole so that overflow toilet water flows out back into toilet tank via the overflow holes. The device further comprises a viscosity indicator, a stickiness indicator, and a color and gray scale calibration image. The camera of the electronics unit looks down on sensors, viscosity indicators, stickiness indicators, Color and gray scale calibration images and captures audios and videos of the toilet water and various other measurable parameters listed somewhere else in this specification. The matte white background and reticle enhances camera ability to determine fluid color, turbidity and detect particulates. The tube nozzle directs fluid parallel to walls causing swirling motion down the sides. The swirling toilet water flows over sensors and indicators and into a lower container. The magnetic stirrer increases mixing through swirling the fluid. The fluid pump inlet pumps fluid from toilet tank water and the fluid pump outlet pumps the toilet fluid into tubing. The multi-speed fluid pump helps increase or decrease pumping speed based on the pumping requirements.

Referring to FIG. 7D, it illustrates a mechanism of bowel fluid flow through the device, according to one or more embodiment. The bowel fluid flow mechanism starts with the toilet water sample entering a pump through a pumping action. The pump then pushes the water into a tubing system. As the toilet water leaves the tube nozzle from the fluid pump at a 90-degree angle, it creates a swirling motion down the sides of the container. This swirling toilet water flows over sensors and indicators located within the container. Subsequently, the water flows into the lower container, where it fills up to just below the Overflow Holes. To ensure the water level remains consistent, a float (not shown in FIG. 7D), helps regulate the height of the toilet water. Any excess water beyond the desired level is vented back into the toilet tank through the Overflow Holes. Furthermore, to enhance mixing in the container, a Magnetic Stirrer is used, which can increase or adjust the mixing intensity as needed during the bowel fluid flow process. This mechanism enables effective sampling and monitoring of toilet water for various purposes, such as analyzing bowel movements and related indicators.

In some embodiments, the device comprises a density and/or a viscosity indicator to measure density and/or viscosity of the bowel fluid. The density and/or the viscosity indicator can either float freely in the lower bowel fluid container or be slidably mounted to the side, depending on whether only density or both density and viscosity are to be measured.

Referring to FIG. 7E, it illustrates viscosity and density measurement mechanism in the device, according to one or more embodiments. To measure density, the device utilizes marks on the density indicator shaft, which are visible to the electronics unit camera. The camera observes the water level on the indicator shaft as an indication of density. As the density of the bowel fluid increases, the density indicator floats higher on the shaft, allowing the camera to precisely determine the density of the fluid. To measure viscosity, the device maintains a set pump flow or magnetic stirrer speed during the process. The swirling speed of the bowel fluid inside the container is proportional to its viscosity. The swirling speed on the density/viscosity indicator is carefully measured by the electronics unit camera, providing valuable information about the fluid's viscosity. By combining these density and viscosity measurements, the device offers a comprehensive analysis of the bowel fluid, facilitating accurate and valuable insights for medical and diagnostic purposes. The integrated technology ensures efficient monitoring and assessment, contributing to improved healthcare management and diagnosis.

In an embodiment, the device comprises a vacuum sample jar to collect a bowel sample for analyte and/or microbiome analysis. The vacuum sample jar conveniently and removably slides into a protective float. Referring to FIG. 7F, it illustrates a device comprising a vacuum sample jar, according to one or more embodiments. The sample collection process begins by opening the sample valve, allowing the sample jar to be filled with bowel fluid. This fluid is suctioned from the bowel fluid container through the sample valve and directly into the vacuum sample jar. Once the sample collection is complete, the sample jar is carefully pulled off the sample valve needle to remove it from the device. This well-designed process ensures hygienic and efficient sample collection for biome analysis. The integration of the vacuum sample jar and the sample valve simplifies the procedure, allowing for precise and controlled sample acquisition. This mechanism guarantees reliable and uncontaminated samples, enabling accurate and insightful analyte and biome analysis for medical and research purposes. The ease of use and reliable sample collection further enhances the overall functionality of the device in supporting healthcare professionals in making informed decisions and improving patient care.

Patient who has higher rates of inadequate bowel preparations include: men, older age, higher BMI, diabetes, use of opioids, previous colorectal surgeries, low socioeconomic status, and patients with limited English proficiency. Nyugen et. al demonstrated in a study of 300 patients in the United States that nearly 90% of patients with poor bowel preparation and failed to either complete the preparation or follow written instructions. However, although non-adherence is an obvious risk factor towards inadequate preparation, this is difficult to measure because it is a self-reported factor.

It is essential that after stool is deemed clear; a final bowel prep is conducted 3-6 hours prior to colonoscopy to remove clear bile/mucus from the colon; thus, another studied risk factor is a “runway” time from last prep to colonoscopy that is greater than 6 hours.

Several randomized controlled trials have demonstrated improvement in colonoscopy prep with use of individualize oral information given by a trained provider, educational videos by a trained provider, and phone call and text message reminders to perform prep. There does not appear to be an iterative smart-phone-based program that guides a patient's bowel prep.

In an embodiment a visual based sensor and/or electronic sensor is utilized to assess the rectal effluent and indicate a readiness level via the devices and systems described herein. The one or more parameters that can be assessed are color, turbidity, chemical composition, stickiness, viscosity, particulate concentration, particulate distribution and acuity.

An embodiment relates to a method to determine the status of colon cleansing in subjects prior to a gastrointestinal procedure. The method comprises obtaining a rectal effluent using a device; capturing an image of the rectal effluent; assessing the image for readiness of bowel preparation; and providing an assessment of bowel preparation to undergo colonoscopy; and wherein the device comprises a first portion comprising an imaging portion configured to captures an image of the rectal effluent and a second portion comprising a stirrer, a suction pump, and a container wherein the second portion is configured to obtain the rectal effluent.

The method can be used to detect any kind of solid substance that has spectral characteristics that can be viewed within at least portions of the fields of view of the image capture array. Individual pixels, or groups of pixels, of captured image information can be analyzed to assess if the pixel(s) display spectral characteristics of the target substance. In embodiments, the method utilizes high-resolution image capturing that allows even minute traces of a target substance to be detected if even only a single pixel in an image corresponding to the target substance is captured.

An embodiment relates to system comprising the device to determine a status of bowel cleansing and a processing system.

In an embodiment, the system comprises a device described in this specification; a communication module configured to communicate with a computing device; and the computing device comprising a processor and a tangible non-transitory memory configured to communicate with the processor, wherein the tangible non-transitory memory has stored thereon instructions executable by the system to cause the system to perform operations comprising: receive a captured image through a receiving component; convert intensity data from at least one of the red channel, the green channel, or the blue channel from at least a portion of an image to a first data point having a first value through a pre-processing module; compare the first data point with the plurality of data points from a standardized curve to identify a first value of an image based assay parameter via an information processing module; predict a cleanliness class and readiness level based on the value of the assay parameter via an information processing module; and provide a recommendation based on the status of the bowel cleansing.

The device and the system are intelligently configured to conduct comprehensive measurements, encompassing various crucial factors in bowel fluid analysis. Viscosity is accurately determined through motion analysis, where the device employs either a maze or mesh to assess the swirling speed of the bowel fluid. Additionally, the device assesses stickiness by considering multiple imaging factors such as color and texture analysis, distinguishing between the jagged shapes associated with the mesh and the smoothed shapes resulting from sticky mucus. Density measurements, as discussed previously, are also an integral part of the analysis, with the device utilizing density indicators to precisely determine the density of the bowel fluid. Furthermore, the device incorporates sophisticated analysis techniques for particulate count and size distribution in the bowel fluid. It effectively identifies and quantifies suspended particles, providing insights into both their count or concentration and size distribution. This comprehensive approach to measurement ensures a thorough and accurate assessment of the bowel fluid, enabling healthcare professionals to gain valuable insights and make informed decisions in medical diagnostics and research.

In an embodiment, the captured image is processed to convert intensity data from at least one of the red channel, the green channel, or the blue channel from at least a portion of an image to a first data point having a first value through a pre-processing module; color information in the digital image is analyzed and compared with the plurality of data points from a standardized curve to identify assay parameter via the image processing module, the assay parameters comprising turbidity and color.

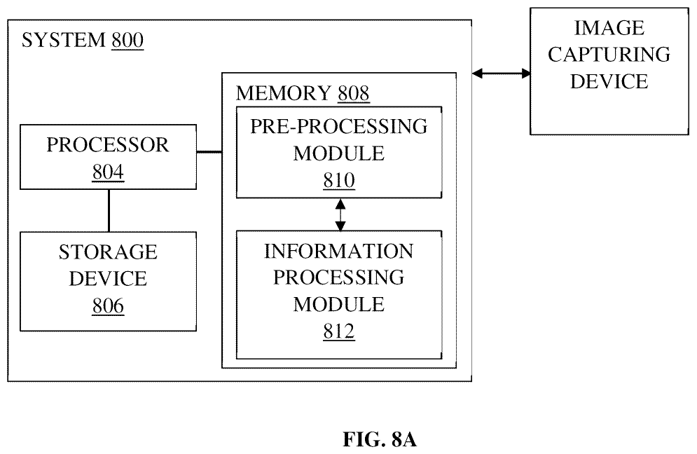

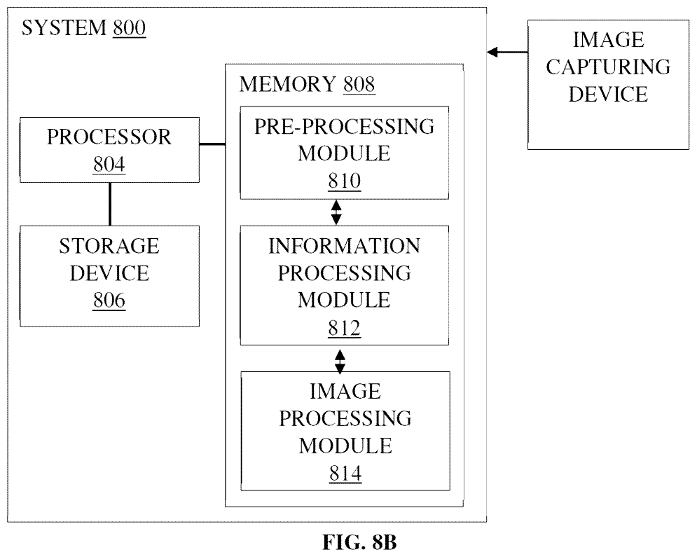

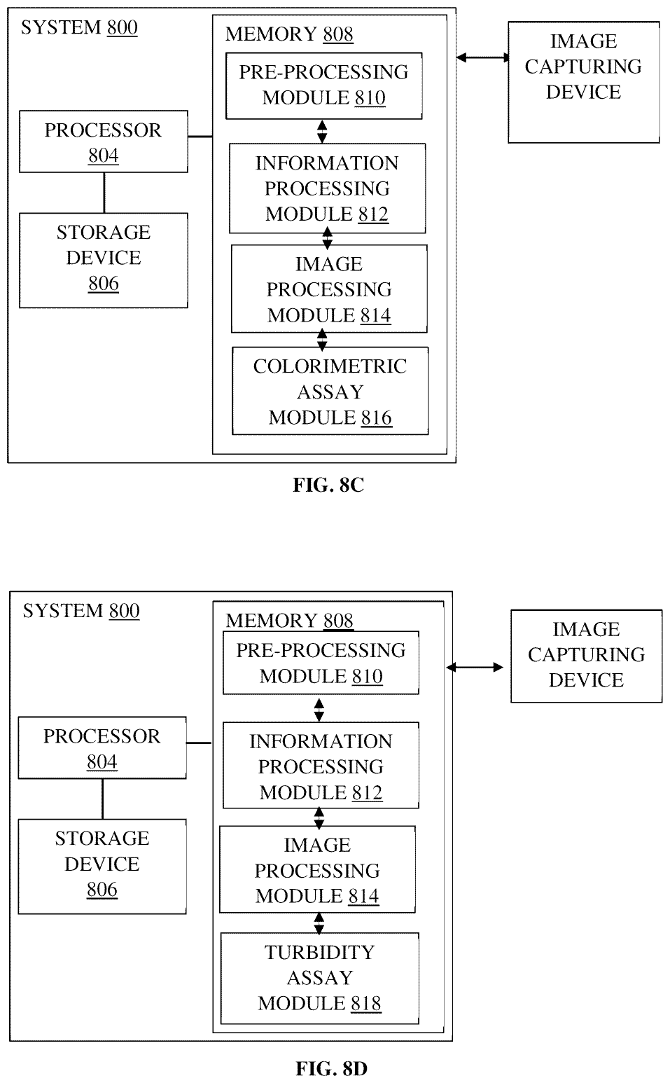

Referring to FIG. 8A, system 800 comprises, device 802, a processor 804, a storage device 806 and a memory 808 further comprising a pre-processing module 810 and an information processing module 812. The information processing module 812 analyzes information captured by the device and provides a recommendation based on the status of the bowel cleansing. The device can send the captured images and other information collected by multiple sensors coupled to the device to the pre-processing module 810 of the system that is remotely located.

In an embodiment, the pre-processing module 810 is configured for data pre-processing. The data pre-processing comprises cleaning, filtering, and transforming the data to prepare it for further processing.

In an embodiment, the pre-processing module 810 comprises a sensor data pre-processing module. The sensor data pre-processing module is configured for sensor data pre-processing such as filtering, calibration, and feature extraction. In another embodiment, the pre-processing module 810 comprises an image pre-processing module. The image pre-processing module is configured for image correction. Image correction involves correcting any distortions or aberrations in the image, such as lens distortion or vignetting. Image correction may also comprise at least one of a pixel brightness transformation, histogram equalization, and sigmoid stretching. The pixel brightness transformation is used for brightness corrections and gray scale transformation. The pixel brightness transformation utilizes one of a power law transform operation, sigmoid stretching and histogram equalization. Histogram enhancement may be used for contrast enhancement and sigmoid stretching may be used for tailoring the amount of lightening and darkening to control the overall contrast enhancement.

In an embodiment, the information processing module 812 is configured for data fusion for combining the image and/or sensor data to create a single, more informative representation of the input. This could be done using techniques such as feature extraction, feature matching, or deep neural networks. In another embodiment, the information processing module 812 utilizes statistical, mathematical, or machine learning techniques to extract meaningful information from the fused data comprising the captured images and other information collected by multiple sensors. The analysis includes tasks such as suspended solid particle detection, turbidity detection, and/or color detection. The information processing module comprises an artificial intelligence unit that uses supervised deep-learning (DL) methods for automated feature learning from raw training images for assessment of readiness of colon preparation and prediction of a time estimate for colon readiness. In yet another embodiment, the information processing module 812 is further configured for data interpretation for drawing conclusions, decision making, and recommendations based on the information. In yet another embodiment, the information processing module 812 is further configured for communicating the results of the data processing and analysis to other systems or people, such as through a report or an API.

In an embodiment, the artificial intelligence unit utilizes computer vision and deep neural network for assessment of colon readiness. The computer vision may comprise an attention module that analyzes the images to identify presence of particles in the rectal effluent with high resolution accuracy. The attention module utilizes an attentional neural network based on a convolutional neural network (CNN) to assess a readiness level of colon preparation.

In an embodiment, the information processing module 812 comprises an image processing module. The image processing module comprises an image analysis software and utilizes the artificial intelligence unit to analyze images received from the imaging device. In some embodiments, the imaging device is integrated into the information processing module. In some embodiments, the processor is one of a tablet computer, a portable computer, and a wearable computer. In some embodiments, the processor is in communication with a clinician device to be in contact with a clinician.

Referring to FIG. 8B, system 800 comprises a device 802 shown in FIG. 8A, a processor 804, a storage device 806 and a memory 808 further comprising a pre-processing module 810, an information processing module 812, and image processing module 814. The information processing module 812 analyzes information captured by the device and provides a recommendation based on the status of the bowel cleansing.

The image analysis module is configured for color space conversion, color normalization and chromaticity estimation. The image analysis module is further configured for post-processing. Color space conversion involves converting the image from its native color space (e.g., RGB, YIQ or CMYK) to a color space that is better suited for chromaticity estimation, such as CIELAB XYZ or CIELAB L*a*b*. The color normalization step involves normalizing the image to account for variations in lighting conditions or camera settings. This can be done by adjusting the overall brightness and contrast of the image or by applying color correction algorithms. Chromaticity estimation involves estimating the chromaticity of the image using a variety of algorithms, such as color histograms, color moments, or color models. Post-processing step involves any additional processing steps, such as filtering or thresholding, to improve the accuracy of the chromaticity estimation.

The image analysis module 814 uses the trained model along with color matching algorithms to calculate a color change. One of the various color spaces, for example RGB, HSV, YIQ, CMYK, and CIELAB 1976 L*a*b* can be used for identifying the color change.

In an example, the color matching algorithm comprises a Correlation Coefficient (CC) method. The CC method calculates correlation coefficients between test image and trained images. The value of the coefficient varies between −1 and 1, where ±1 indicates the strongest possible agreement and 0 the strongest possible disagreement.

In an example, the color matching algorithm comprises a deltaE method. Delta E levels are the difference between the displayed color and the original color standard of the input content. Lower Delta E figures indicate greater accuracy, while high Delta E levels indicate a significant mismatch. The deltaE method is based on measuring color differences between two images using deltaE (ΔE*) distance metric obtained from the CIELAB 1976 L*a*b* color difference formula

$$Δ {E}^{*} = \sqrt{{( Δ {L}^{*} )}^{2} + {( Δ {a}^{*} )}^{2} + {( Δ {b}^{*} )}^{2}}$$

wherein,
L*, a*, and b* are dimensions of the CIELAB L*a*b* color space;
L* axis represents lightness in the range of black (0) to white (100);
a* axis varies over red (+a*) to green (−a*)
b* axis describes yellow (+b*) to blue (−b)
ΔE* calculates a lower score for the similar images as the distance between similar images is smaller than the distance between dissimilar images.

In some embodiments, the image analysis module evaluates the image obtained by the imaging component. The image analysis module may assess the status of bowel cleansing from the rectal effluent. In some embodiments, the image analysis software may assess the observable edges or contrasts (e.g., a flushable marker) on the bottom of, or in, the seat of toilet commode.

The image analysis software may include the ability to transform the images provided by the subject, creating enhanced images which may include highlighting, coloring, emphasizing or de-emphasizing of detail, and digital filtering among many other potential transformations. For example, the image analysis software may subtract out the background image without the bowel effluent or subtract out other foreign objects or abnormalities, such as toilet paper, glare, or shadows, from the bowel effluent image. The image analysis software may transform the image to align and correct the image to resemble the background image in brightness/contrast, size, and orientation (e.g., pan, tilt and rotation). Multiple software programs may be utilized together in order to fully analyze the image. In an embodiment, a capture setting can be controlled through image processing module 814 to image each rectal effluent at multiple exposures for multiple measurements of each rectal effluent. Image segmentation is performed on the original image taken by the camera through an algorithm model to obtain several sample images. In an embodiment, the reference image is a circular image selected from within the sample image.

The image analysis software may also generate results and reports based on the analysis of the image. In some embodiments, the artificial intelligence unit alters protocols or displays instructions based on the image analysis evaluation of the bowel effluent (e.g., utilizing a machine learning process). In some embodiments, the artificial intelligence unit provides real-time feedback of the status of the colon cleanse to the subject based on the image analysis software. Consequently, the artificial intelligence unit facilitates customization of protocols and prep medication dosing (e.g., increase or decrease in amount or frequency, continuation of dosing regimen, termination of dosing regimen) in real-time, based on active monitoring of the images unlike convention preparations utilizing a universal approach without a form of monitoring. In some embodiments, the artificial intelligence unit instructs the information processing module to report the image results to a clinician device.

An embodiment relates to colorimetric assay of the image captured by the device using the system comprising a computer program. The automated colorimetric analysis is based on feature matching and a region-based convolutional neural network that is able to detect a presence of solid particles with a wide variety of light conditions, backgrounds and perspectives.

Referring to FIG. 8C, the system 800 comprises, the device 802 shown in FIG. 8A, a processor 804, a storage device 806 and a memory 808 further comprising a pre-processing module 810, an information processing module 812, image processing module 814 and a colorimetric analysis module 816.

The colorimetric assay comprises obtaining an image of the assay, optionally correcting for ambient lighting conditions in the image, converting the RGB intensity data to a first data point, recalling a predetermined standardized curve, comparing the first data point with the standardized curve, and identifying the value for the assay parameter from the standardized curve. The assay also compensates for differences in lighting conditions when taking images under uncontrolled lighting conditions. Embodiments of the methods described herein allow for the use of a fully integrated imaging sensor, such as a CMOS sensor or CCD sensor, to accurately analyze a colorimetric assay.

Embodiments of the present method convert the intensity data from at least one of the red channel, the green channel, or the blue channel (“RGB”) from at least a portion of the image of the first colorimetric assay to a data point having a first value and a second value that indicate the color of the test colorimetric assay.

In one embodiment, the RGB intensity data is converted to a data point using the CIELAB color space to code the colorimetric image to overcome inadequacies of the simple RGB analysis. The CIELAB system is the most recognized method in which color is represented, with tristimulus values X, Y, and Z characterizing the emission color of luminescence data across the wavelength range of visible light. In an example, the CIELAB 1931 xyY system is used in which the Y parameter represents luminance (brightness) of a color and derived parameters x and y determine the chromaticity of a color. Conversion of the obtained image data into the CIELAB 1931 xyY color space involves three steps, in which the color space terms are derived from the conventional RGB values. In devices that utilize the sRGB color standard, such as digital cameras, cameras in mobile devices such as mobile phones, tablets, and portable computers, the nonlinear sRGB values are converted to linear RGB values using

$${C}_{linear} = {\{ \frac{Csrgb + 0.055}{1.055} \}}^{2.4}$$

in which C_{srgb} stands for R_{srgb}, G_{srgb}, and B_{srgb}, and Cₗᵢₙₑₐᵣ indicates Rₗᵢₙₑₐᵣ, Gₗᵢₙₑₐᵣ, and Bₗᵢₙₑₐᵣ. Then, the linear RGB values can be converted to tristimulus values X, Y, and Z using

$$[ \begin{matrix}X \\ Y \\ Z\end{matrix} ] = [ \begin{matrix}0.4124 & 0.3576 & 0.1805 \\ 0.2126 & 0.7152 & 0.0722 \\ 0.0193 & 0.1192 & 0.9505\end{matrix} ] [ \begin{matrix}{R}_{linear} \\ {G}_{linear} \\ {B}_{linear}\end{matrix} ]$$

Finally, the chromaticity-values x and y are obtained by

$$\{ \begin{matrix}x = \frac{X}{X + Y + Z} \\ y = \frac{Y}{X + Y + Z}\end{matrix} \}$$

The new color space specified by x, y and Y is represented in a 2-D diagram—the Horseshoe shaped Chromaticity diagram. The pure colors are located on the boundary curve from blue (380 nm) to red (700 nm), while all the mixed colors, such as yellow and pink, are represented within the area enclosed by the curve. The position of a point in the diagram indicates the chromaticity of the corresponding color. In practice, the first and second values of the data point from the colorimetric assay correspond with the x and y coordinates of the image as plotted on the xy chromaticity diagram.

The xy chromaticity diagram of the CIELAB 1976 color space system can be used to predict the outcome of a mixture of two colors. The mixed color lies along the straight line connecting the two points of the original colors on the xy chromaticity diagram. The ratio of the two original colors determines the position of the mixed color. This can be potentially useful in more complicated colorimetric assays. Notably, the hue and saturation of a color, based on which the widely used HSV and HSL models were defined, can be derived from its location on the xy diagram. Considering these assets of the CIELAB 1931 xyY color space, the present method of analyzing colorimetric assays to quantify the colors is versatile and works well as demonstrated in the examples discussed below.

The first data point is compared with a predetermined three-dimensional standardized curve. The standardized curve includes a plurality of data points wherein each data point has an x-value, a y-value, and a z-value. The x- and y-values correspond with the x and y-values of the xy chromaticity diagram. The z-value corresponds with a predetermined assay-value, such as, for example, an analyte concentration. An exemplary predetermined standardized curves is generated with analytes over a range of known concentrations by obtaining images of a plurality of colorimetric assays conducted with known analyte concentrations, converting the RGB intensity data for each known analyte concentration to a data point having an x-value and a y-value indicative of the chromaticity of the data point and plotting the x-value and the y-value along with the z-value, indicative of the analyte concentration, on a three dimensional curve. The standardized curve includes not only the data points obtained from the assays conducted with known analyte concentrations but also the data points along the curve connecting the data points from the known analyte concentration assays. The standardized curve may be prepared in advance and stored in a storage medium, such as the device's RAM and writable media, from which it is recalled for use in analyzing a test assay.

In some circumstances, the obtained image of the test assay may include areas having different ambient lighting conditions, such as a portion of the image may be in a shadow or exposed to brighter lighting conditions. Embodiments of the system may recognize and account for changes in lighting conditions across the image. For example, if the measure intensity of one or more reference areas does not have a linear relationship with the remainder of the reference areas, then this would indicate that those references areas are exposed to different ambient lighting conditions. The method of image analysis for colorimetric assay of an image is described in U.S. Pat. No. 9,506,855B2, which is incorporated herein by reference.

An embodiment relates to turbidity assay of the image captured by the device using the system comprising a computer program. The system comprises a turbidity detection module that is configured to execute turbidity detection based on the RGB model.

Referring to FIG. 8D, the system 800 comprises, the device 802 shown in FIG. 8A, a processor 804, a storage device 806 and a memory 808 further comprising a pre-processing module 810, an information processing module 812, image processing module 814 and a turbidity assay module 818.

The turbidity detection based on the RGB model comprises the following steps:
Step 1: Defining region of interest for a captured image and record it as a sample image; the sample image also includes at least one reference image for comparison; wherein, the area where the reference image is located is an image of a pure dark block. It should be understood that the reference image and the sample image may have any shape. In an embodiment, the reference image and the sample image are circular images to ensure as much as possible the uniformity of light when the light illuminates the reference or sample images.
Step 2: Obtaining the three-channel components of a single pixel point Nⁿ in the surrounding area of the reference image under the RGB model, and the three-channel component of a single pixel point Mⁿ in the reference image area superimposed with the light-permeable solution scattering effect of the rectal effluent. Specifically, the three-channel components of a single pixel point Nⁿ in the surrounding area of the reference image under the RGB model are obtained and denoted as Y_{N}ⁿ (R, G, B); and the three-channel component of a single pixel Mⁿ in the reference image area superimposed with the light-permeable solution scattering effect of the rectal effluent to be tested is obtained under the RGB model and denoted as Y_{M}ⁿ (R, G, B).
Step 3: The brightness values V_{R}, V_{G}, and V_{B} of each channel of Y_{N}ⁿ (R, G, B) and Y_{M}ⁿ (R, G, B) is obtained and the maximum value of V_{R}, V_{G}, and V_{B} at the corresponding pixel point is recorded. Also, the brightness values V_{R}, V_{G}, V_{B} of each channel of Y_{N}ⁿ (R, G, B), are obtained and the maximum value of V_{R}, V_{G}, V_{B} as the brightness value V_{N}ⁿ of the pixel is recorded.
Step 4: In the surrounding area of the reference image, brightness value group is {V_{N}ⁿ} for plurality of pixels {Nⁿ}, and in the reference image area brightness value group is {V_{N}ⁿ} for plurality of pixels {Mⁿ}. V₀ is a representative brightness value for a minimum brightness point and V is a representative brightness value for a maximum brightness point in the brightness value group {V_{M}ⁿ}. In an example, V is the arithmetic mean value of the brightness value group {V_{M}ⁿ} and the reference brightness value V₀ is the arithmetic mean value of the brightness value group {V_{N}ⁿ}. Specifically, the surrounding area of the reference image is composed of several pixels, which are respectively denoted as pixel points N¹ and N², N³, N⁴ until Nⁿ; pixels cover all the pixels in the area around the reference image. The brightness values of all pixels in the area are obtained as in step 3. In some embodiments, the size comparison of all the pixel point values V_{N}ⁿ is done in the area to measure the brightness value V₀ in the area surrounding the reference image. Specifically, the brightness value corresponding to N¹ is VN¹, the brightness value corresponding to N² is VN², and the brightness value corresponding to Nⁿ is VN^{m}. VNⁿ is sorted by size to obtain the maximum or minimum value of a single pixel in the area and is used to characterize the brightness value of the area around the reference image.

In other embodiments, the arithmetic average value VNⁿ of all the pixels in the area is calculated, and the average value is used as the brightness value in that area. Using the average value as the brightness value can improve the accuracy of the brightness value in the area. Specifically, if the surrounding area of the reference image includes pixels N¹, N², N³, N⁴ and Nⁿ, the brightness value of the surrounding area of the reference image is calculated by (VN¹+VN²+VN³+ . . . +VNⁿ)/n.

The brightness value VMⁿ of several pixels in the reference image is obtained and the brightness values of the several pixels are sorted to calculate the maximum/minimum value in VMⁿ. Alternatively, the arithmetic average of the brightness values VMⁿ of several pixels is calculated to obtain V. The image in the reference image is composed of several pixels, which are respectively denoted as pixel points M¹, M², M³, M⁴ and Mⁿa; 1-n pixels cover all pixels in the reference image. The brightness values of all pixels are obtained by obtaining several brightness values VMⁿ of the pixel points corresponding to the target pixel. In some embodiments, all the pixel values VMⁿ in the area obtained are compared in size to obtain the value Mⁿ in VMⁿ. The maximum or minimum value is used to measure the brightness value V of the area in the reference image. Specifically, the brightness value corresponding to M¹ is VM1, the brightness value corresponding to M² is VM2, and the brightness value corresponding to Mⁿ is V_{M}ⁿ. The brightness values are sorted to obtain the maximum and minimum value of a single pixel in the region, and accordingly the brightness value of the region of reference is calculated. In some embodiments, the obtained value VMⁿ of all pixels in the region is calculated as Arithmetic average value. Specifically, the reference image includes pixels M¹, M², M³, M⁴ and up to Mn. The brightness value of the area is calculated by (V_{M}¹+V_{M}²+V_{M}³+ . . . +V_{M}ⁿ)/n.
Step 5: The turbidity value t of the rectal effluent is obtained using as the ratio of V₀ and V. In some embodiments, the turbidity value τ of the rectal effluent is obtained by substituting the brightness value of plurality of pixel points in the surrounding (i.e., V) and the brightness value of plurality of pixel points in the reference (i.e., V₀) into the formula:

$$τ = \frac{1}{b} {e}^{{a}^{\frac{v}{{v}_{0}}}}$$

where b is the optical path and a is the coefficient related to the clear rectal effluent.

In an embodiment, the device comprises a spectrophotometer for absorption and or emission based rectal effluent analysis to determine the status of colon preparation.

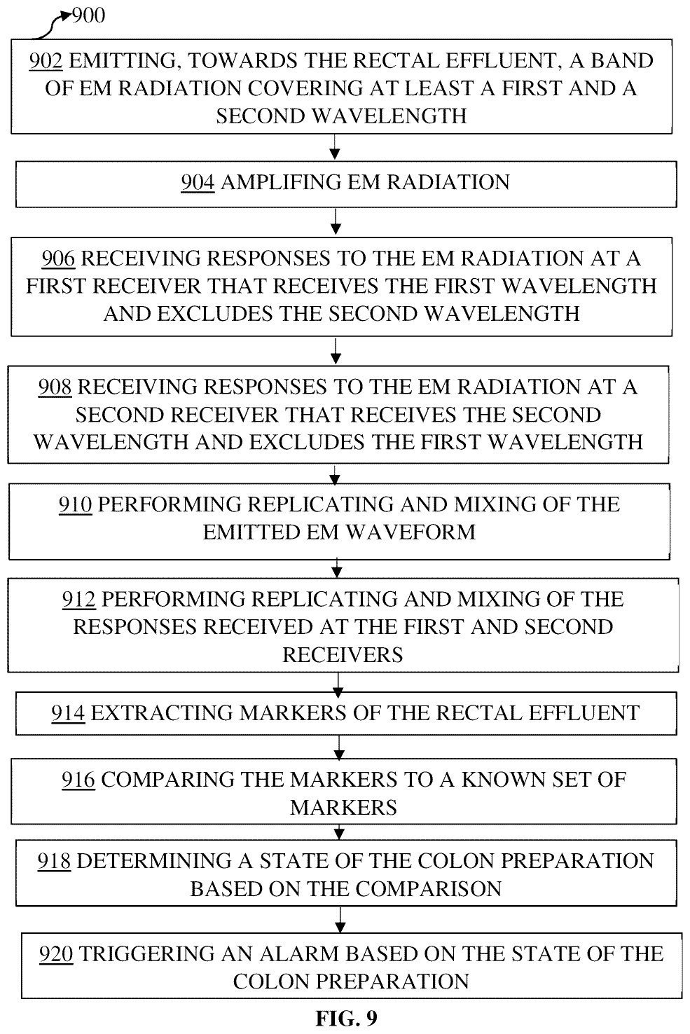

Referring to FIG. 9, it illustrates a method for assessing readiness of colon preparation, according to an embodiment. The method 900 comprises the following steps:
Step 902, of the method comprises emitting, towards the rectal effluent, a band of electromagnetic (EM) radiation covering at least a first and a second wavelength.
Step 904, of the method further comprises amplifying EM radiation.
Step 906, of the method further comprises receiving responses to the EM radiation at a first receiver that receives the first wavelength and excludes the second wavelength.
Step 908, of the method further comprises receiving responses to the EM radiation at a second receiver that receives the second wavelength and excludes the first wavelength.
Step 910, of the method further comprises performing replicating and mixing of the emitted EM waveform.
Step 912, of the method further comprises performing replicating and mixing of the responses received at the first and second receivers.
Step 914, of the method further comprises extracting markers of the rectal effluent.
Step 916, of the method further comprises comparing the markers to a known set of markers.
Step 918, of the method further comprises determining a state of the colon preparation based on the comparison.
Step 920, of the method further comprises triggering an alert based on the state of the colon preparation.

In an embodiment of the system, the device further comprises a sensor. In another embodiment, the sensor comprises at least one of a colorimeter, spectrophotometer and a turbidimeter. In yet another embodiment, the system is further operable to: receive data collected via the one or more sensors through the receiving component; process the data collected via the one or more sensors to filter a noise present in the data and get a normalized dataset; compare the normalized dataset with a standardized model to identify a second value of a sensor-based assay parameter via the information processing module; correlate the first value with the second value; and generate a percentage level for the cleanliness class and the readiness level.

In an embodiment, the device utilizes SpectralFusion for colon prep analysis. SpectralFusion is a proprietary technology developed by Trinamix® that combines two different types of sensors to capture and analyze both visible and invisible light. Specifically, SpectralFusion combines a near-infrared (NIR) sensor and a conventional color camera sensor. The NIR sensor detects light that is outside the visible spectrum, which provides additional information about the chemical composition of the object being analyzed. The color camera sensor captures visible light, providing details about the object's appearance and texture. By combining these two sensors and analyzing the data together, Spectral Fusion is able to extract more comprehensive information about an object, such as its chemical composition, surface quality, and texture, than either sensor could provide alone.

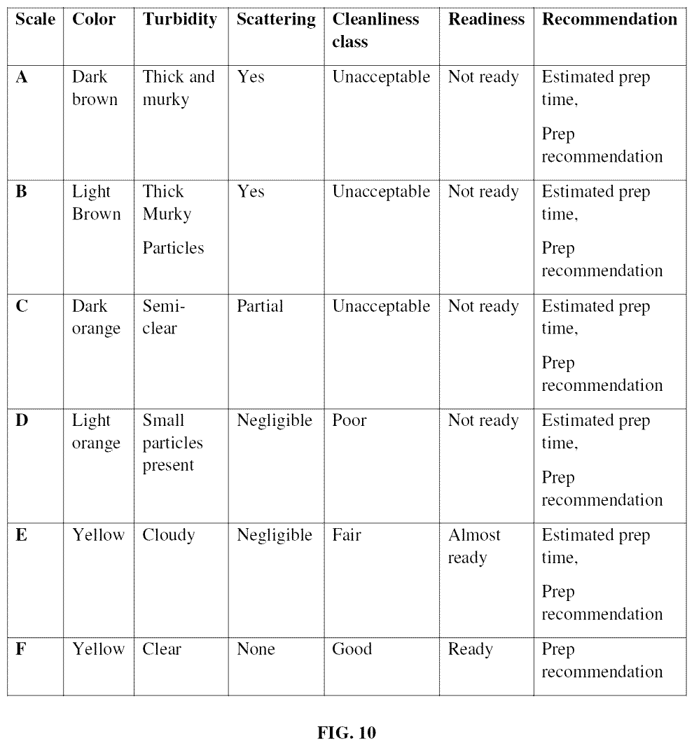

Referring to FIG. 10, it describes a table for scale and readiness analysis via colonoscopy readiness device, according to an embodiment. In an example, Scale A comprises rectal effluent that is dark brown, thick, and murky and scatters EM radiation falling on it. This scale is unacceptable as the colon is not ready. In an example, Scale B comprises rectal effluent that is light brown, thick and murky with particles. It scatters EM radiation falling on it. This scale is unacceptable as the colon is not ready. In an example, Scale C comprises rectal effluent that is dark orange and semi-clear. It scatters EM radiation falling on it partially. This scale is unacceptable as the colon is not ready. In an example, Scale D comprises rectal effluent that is light orange and has presence of small particles. There is negligible scattering of EM radiations. This scale indicates poor cleanliness of rectum, and the colon is not ready for colonoscopy at this stage. In an example, Scale E comprises rectal effluent that is yellow and cloudy. There is negligible scattering of EM radiations. This scale indicates fair cleanliness of rectum, and the colon is almost ready for colonoscopy at this stage. In an example, Scale F comprises rectal effluent that is yellow and clear. There is no scattering of EM radiations. This scale indicates good cleanliness of rectum, and the colon is ready for colonoscopy.

In an embodiment, the patient user interface of the mobile application will involve several steps for efficient and accurate testing. Initially, the patient will confirm the QR code or verification code found in their test kit comprising the device to establish a secure connection. The mobile application is configured to associate the identification number of the kit with the patient and take a record of it into the database. The patient's user interface will allow the patient to scan the identification number of the device. In some embodiments, before beginning the test, the patient will be required to provide their demographics, including age, height, gender, weight, and medical history, if not already completed. They will then be able to record the time and date of taking the prep medicine, as well as specify the type of prep medicine used. The patient will have the capability to capture a photograph of their stool, with relevant image information being transmitted alongside it. In some embodiments, in the event that the system finds the image unsatisfactory, the patient will be prompted to resend it via the mobile application. This process will repeat for each prep medicine taken and subsequent bowel movements. Once all images have been sent, the patient will receive a confirmation screen and a confirmation email, acknowledging the completion of the testing process.

In an embodiment, the provider's user interface of the mobile application is configured to provide comprehensive features for monitoring and accessing patient data. In some embodiments, the doctor is enabled to suggest preparation or medication dosing and create dosing schedules based on individual patient requirements. In some embodiments, the doctor is enabled to confirm or cancel colonoscopy appointments based on the patient's data and progress. In some embodiments, an interface is configured to allow the doctor to securely message the patient, facilitating effective communication.

The doctor will have access to relevant colonoscopy metrics and industry standards via the user interface, enabling informed decision-making and analysis. Through the patient dashboard, the doctor will be able to review all patients and access their respective colonoscopy history for comprehensive patient management. In some embodiments, the provider's user interface allows the provider to scan the identification number of the device. In some embodiments, the provider's user interface allows the provider to manually enter the identification number of the device into a database. The mobile application is configured to associate the identification number to with the subject and take a record of it into the database.

In an embodiment, the assessment via the device may be remotely supervised and verified by a remote healthcare provider using a networked environment. the Software can be one of a web-based platform, a mobile application and a VPN platform.

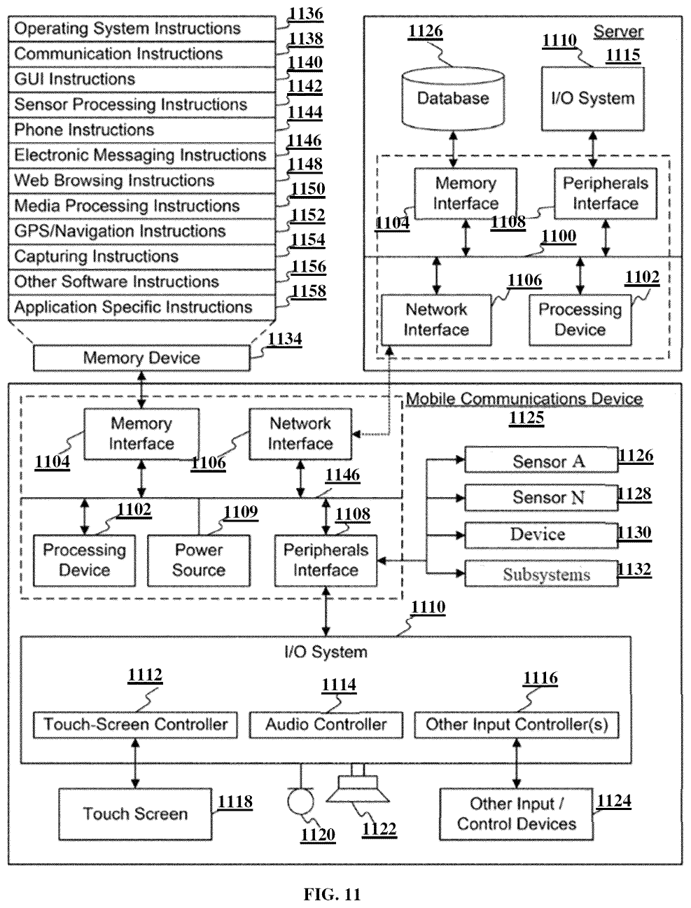

FIG. 11 is an exemplary block diagram illustrating some of the components of the exemplary system that uses image data capture by mobile communications devices for medical testing, consistent with the present disclosure. In one embodiment, server 1125 and mobile communications device 1125 directly or indirectly accesses a bus 1110 (or other communication mechanism) that interconnects subsystems and components for transferring information within server 1115 and/or mobile communications device 1125. For example, bus 1100 may interconnect a processing device 1102, a memory interface 1104, a network interface 1106, a peripherals interface 1108 connected to I/O system 1110, and power source 1109.

Processing device 1102, shown in FIG. 11, may include at least one processor configured to execute computer programs, applications, methods, processes, or other software to perform embodiments described in the present disclosure. For example, the processing device may include one or more integrated circuits, microchips, microcontrollers, microprocessors, all or part of a central processing unit (CPU), graphics processing unit (GPU), digital signal processor (DSP), field programmable gate array (FPGA), or other circuits suitable for executing instructions or performing logic operations. The processing device may include at least one processor configured to perform functions of the disclosed methods such as a microprocessor manufactured by Intel™. The processing device may include a single core or multiple core processors executing parallel processes simultaneously. In one example, the processing device may be a single core processor configured with virtual processing technologies. The processing device may implement virtual machine technologies or other technologies to provide the ability to execute, control, run, manipulate, store, etc., multiple software processes, applications, programs, etc. In another example, the processing device may include a multiple-core processor arrangement (e.g., dual, quad core, etc.) configured to provide parallel processing functionalities to allow a device associated with the processing device to execute multiple processes simultaneously. It is appreciated that other types of processor arrangements could be implemented to provide the capabilities disclosed herein.

In some embodiments, processing device 1102 may use memory interface 11204 to access data and a software product stored on a memory device or a non-transitory computer-readable medium. For example, server 1115 may use memory interface 1104 to access database 1126. As used herein, a non-transitory computer-readable storage medium refers to any type of physical memory on which information or data readable by at least one processor can be stored. Examples include random access memory (RAM), read-only memory (ROM), volatile memory, non-volatile memory, hard drives, CD ROMs, DVDs, flash drives, disks, any other optical data storage medium, any physical medium with patterns of holes, a RAM, a PROM, and EPROM, a FLASH-EPROM or any other flash memory, NVRAM, a cache, a register, any other memory chip or cartridge, and networked versions of the same. The terms “memory” and “computer-readable storage medium” may refer to multiple structures, such as a plurality of memories or computer-readable storage mediums located within mobile communications device 1125, server 1115, or at a remote location. Additionally, one or more computer-readable storage mediums can be utilized in implementing a computer-implemented method. The term “computer-readable storage medium” should be understood to include tangible items and exclude carrier waves and transient signals.

Both mobile communications device 1125 and server 1145 may include network interface 1106 coupled to bus 1100. Network interface 1106 may provide two-way data communications to a network. In FIG. 11, the wireless communication between mobile communications device 1125 and server 1115 is represented by a dashed arrow. In one embodiment, network interface 1106 may include an integrated services digital network (ISDN) card, cellular modem, satellite modem, or a modem to provide a data communication connection over the Internet. As another example, network interface 1106 may include a wireless local area network (WLAN) card. In another embodiment, network interface 1106 may include an Ethernet port connected to radio frequency receivers and transmitters and/or optical (e.g., infrared) receivers and transmitters. The specific design and implementation of network interface 1106 may depend on the communications network(s) over which mobile communications device 1125 and server 1115 are intended to operate. For example, in some embodiments, mobile communications device 1125 may include network interface 11206 designed to operate over a GSM network, a GPRS network, an EDGE network, a Wi-Fi or WiMAX network, and a Bluetooth® network. In any such implementation, network interface 1106 may be configured to send and receive electrical, electromagnetic or optical signals that carry digital data streams representing various types of information.

Both mobile communications device 1125 and server 1115 may also include peripherals interface 1108 coupled to bus 1100. Peripherals interface 1108 may be connected to sensors, devices, and subsystems to facilitate multiple functionalities. In one embodiment, peripherals interface 1108 may be connected to I/O system 1110 configured to receive signals or input from devices and to provide signals or output to one or more devices that allow data to be received and/or transmitted by mobile communications device 1125 and server 1115. In one example, I/O system 1110 may include a touch screen controller 1112, audio controller 1114, and/or other input controller(s) 1116. Touch screen controller 1112 may be coupled to a touch screen 1118. Touch screen 1118 and touch screen controller 1112 may, for example, detect contact, movement or break thereof using any of a plurality of touch sensitivity technologies, including but not limited to capacitive, resistive, infrared, and surface acoustic wave technologies as well as other proximity sensor arrays or other elements for determining one or more points of contact with the touch screen 1118. Touch screen 1118 may also, for example, be used to implement virtual or soft buttons and/or a keyboard. While touch screen 1118 is shown in FIG. 11, 1/O system 1110 may include a display screen (e.g., CRT or LCD) in place of touch screen 11218. Audio controller 1114 may be coupled to a microphone 1120 and a speaker 1122 to facilitate voice-enabled functions, such as voice recognition, voice replication, digital recording, and telephony functions. The other input controller(s) 1116 may be coupled to other input/control devices 1124, such as one or more buttons, rocker switches, thumbwheel, infrared port, USB port, and/or a pointer device such as a stylus.

With regard to mobile communications device 1125, peripherals interface 1108 may also be connected to plurality of sensors (Sensor A, . . . , Sensor N) to facilitate image capturing, and measuring of various relevant features. Other sensors (not shown) may also be connected to the peripherals interface 1108, such as a temperature sensor, a biometric sensor, or other sensing devices to facilitate related functionalities. In addition, a GPS receiver may also be integrated with, or connected to, mobile communications device 1125, such as GPS receivers typically integrated into mobile communications devices. Alternatively, GPS software may permit a mobile communications device to access AN external GPS receiver (e.g., connecting via a serial port or Bluetooth).

Consistent with the present disclosure, mobile communications device 1125 may use memory interface 1104 to access memory device 1134. Memory device 1134 may include high-speed random-access memory and/or non-volatile memory such as one or more magnetic disk storage devices, one or more optical storage devices, and/or flash memory (e.g., NAND, NOR). Memory device 1134 may store an operating system 1136, such as DARWIN, RTXC, LINUX, IOS, UNIX, OSX, WINDOWS, or an embedded operating system such as VxWorks. The operating system 1136 may include instructions for handling basic system services and for performing hardware-dependent tasks. In some implementations, the operating system 1136 may be a kernel (e.g., UNIX kernel).

Memory device 1134 may also store communication instructions 1138 to facilitate communicating with one or more additional devices, one or more computers and/or one or more servers. Memory device 1134 may include: graphical user interface instructions 1140 to facilitate graphic user interface processing; sensor processing instructions 1142 to facilitate sensor-related processing and functions; phone instructions 1144 to facilitate phone-related processes and functions; electronic messaging instructions 1146 to facilitate electronic-messaging related processes and functions; web browsing instructions 1148 to facilitate web browsing-related processes and functions; media processing instructions 1150 to facilitate media processing-related processes and functions; GPS/navigation instructions 1152 to facilitate GPS and navigation-related processes and instructions; capturing instructions 1154 to facilitate processes and functions related to image sensor 1126; and/or other and software instructions 1156 to facilitate other processes functions. Memory device 1134 may also include application specific instructions 1158 to facilitate a process for guiding user on the steps of the medical testing. For example, application specific instructions 1158 may cause display of a massage indicative of image insufficiency for medical testing.

Each of the above identified instructions and applications may correspond to a set of instructions for performing one or more functions described above. These instructions need not be implemented as separate software programs, procedures, or modules. Memory device 1134 may include additional instructions or fewer instructions. Furthermore, various functions of mobile communications device 1125 may be implemented in hardware and/or in software, including in one or more signal processing and/or application specific integrated circuits. For example, mobile communications device 1125 may execute an image processing algorithm to identify objects in a received image. In addition, the components and arrangements shown in FIG. 11 are not intended to limit the disclosed embodiments. As will be appreciated by a person skilled in the art having the benefit of this disclosure, numerous variations and/or modifications may be made to the depicted configuration of server 1115. For example, not all components may be essential for the operation of server 1115 in all cases. Any component may be located in any appropriate part of server 1115, and the components may be rearranged into a variety of configurations while providing the functionality of the disclosed embodiments. For example, some servers may not include all of the elements in I/O system 1110.

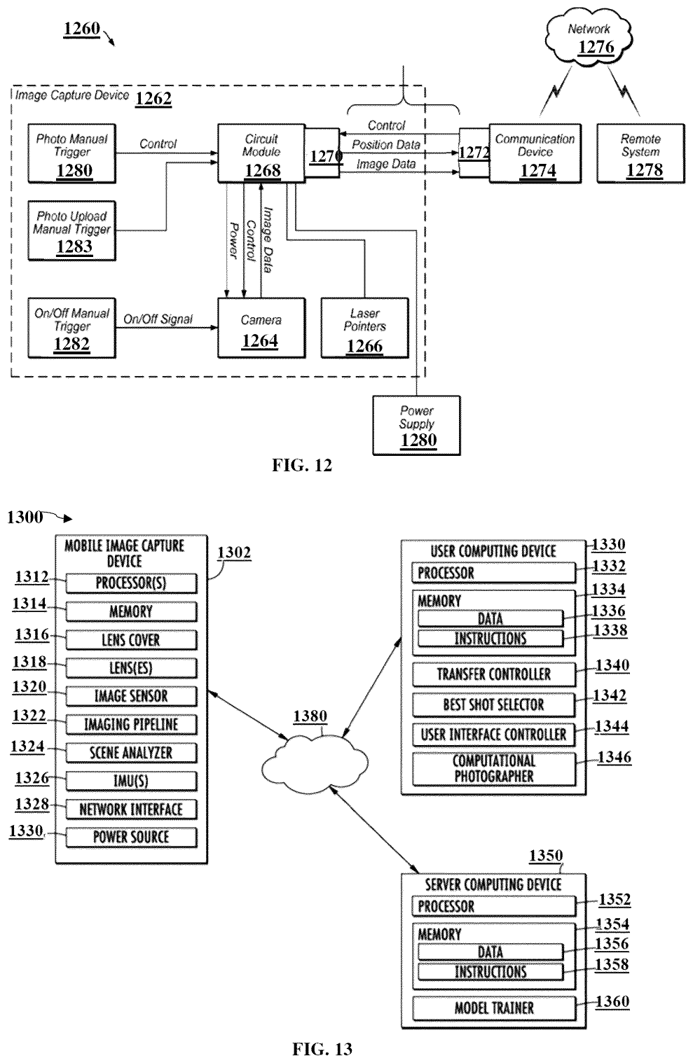

Referring to FIG. 12, it is a schematic diagram illustrating one embodiment of a system including an image capture device with a wireless transmitter/receiver. The system 1260 is comprised of an image capture device 1262, network communication device 1274, network 1276, and remote server system 1278. All internal components of the above components are included in the system.

Digital image capture device 1262 can capture, manipulate, analyze, and transfer digital image data. An example image capture device could be, but is not limited to, a smart phone. 1262 includes digital camera 1264, laser pointer 1266, photo manual trigger 1280, photo upload manual trigger 1283, on/off manual trigger 1282, power supply 1280, and circuit module 1268. Photo manual trigger 1280, photo upload manual trigger 1283, and on/off manual trigger 1282 cause image data to be captured, image data to be uploaded to the remote server system 1278 and turn the image capture device on or off respectively. All triggers are fire responsive to a user interaction such as the press of a button or a tap on a screen. Circuit module 1268 may provide and control power to camera 1264 and laser pointer 1266. Circuit module 1268 includes wireless transmitter/receiver 1270. It is also responsible for doing a necessary image analysis and sending data to communication device 1274. Wireless transmitter/receiver 1270 may transmit and receive information to and from a corresponding transmitter/receiver 1272 on communication device 1274 over wireless connection 1275. Communication device 1274 may be, for example, a personal computer system near a workstation where image capture device 1262 is being used, or a wireless network hub. The network 1276 is a WAN network, such as the internet, through which data from the image capture device will be sent to remote server system 1278. The remote server system 1278 will perform detailed analysis of the data received to determine how the patent should modify their colonoscopy prep, and these modifications will be sent back over the network 1276 and to the image capture device 1274 the same way it was sent.

Referring to FIG. 13, it illustrates a block diagram of the system according to one or more embodiments. System 1300 comprises a Mobile Image Capture device 1302, an Image pipeline component 1322, and Scene analyzer 1324. The Mobile Image Capture device 1302 automatically manipulates and analyzes digital images that it captures. It houses a processor 1312 responsible for image data manipulation and initiates the process of sending image data through network 1380 to User Computing device 1330. Image data is stored in Random Access Memory (RAM) 1314 for rapid access by the processor. The device includes lens 1318 (or lenses) and sensors 1320 for capturing digital image data. The Image pipeline 1322 component encompasses one or more image processing elements to process the raw data received from the image sensor 1320. The Scene analyzer 1324 assesses the desirability of a scene depicted by an image captured by the mobile image capture device 1302. Based on this assessment, it determines whether to store the image or discard it along with other contemporaneously captured images. In some embodiments, the system further comprises an Inertial Measurement Unit (IMU) 1326. The IMU provides orientation, velocity, and gravitational force data applied to the sensor. It plays a role in determining when the image capture device 1302 captures an image upon the closure of a toilet seat. In some embodiments of the system, Network Interface 1328 facilitates the transmission of image data captured by device 1302 through the network 1380, which may be a Wide Area Network like the internet.

In some embodiments of the system, a User Computing device 1330 receives image data from the image capture device 1302 and performs additional analysis, allowing users to add supplementary data before forwarding everything to Server Computing device 1350. In some embodiments of the system, a processer 1332 is responsible for checking whether any image analysis or extra processing is required. It also handles user-input data and initiates processes to send data to the Server Computing device 1350. In some embodiments of the system, a memory device 1334 (specifically, RAM) stores all data to be analyzed or manipulated and programming instructions to execute said analysis or manipulation. In some embodiments of the system, a computational photographer component 1346 performs various image editing and synthesis techniques on received images. It can execute advanced image processing techniques like super-resolution, hyper-lapse, texture mapping, depth mapping, and view synthesis. Adjustable controls for ISO, depth of field, stabilization, and other image parameters are also possible. In some embodiments of the system, server 1350 located in remote conducts the bulk of image analysis and determines necessary modifications to the preparation routine based on the image data sent by the User Computing device 1330. It communicates these instructions back to the User Computing device through network 1380. In some embodiments of the system, a programmable processor 1352 handles image processing to determine modifications in the preparation routine. It may utilize an AI model for more precise analysis, and continual use improves the accuracy of analysis and image recognition. In some embodiments of the system, RAM 1354 component holds data to be analyzed and instructions for processor 1352. In some embodiments of the system, a model trainer module 1360 is used for training the AI employed by processor 1352. Continuous use of the model trainer improves the accuracy of image data analysis and recognition.

FIG. 14 is a flowchart of an exemplary process for completing a colon assessment according to embodiments of the present disclosure. In some embodiments, the exemplary process is executed by different components of system 1400. For example, care provider 1420, information analysis unit 1440, and user 1410. In one embodiment, any action performed by server 1445 may be performed by any combination of mobile communications device 1415, mobile communications device 1425, communications device 1465, and communications device 1475.

Example process starts when healthcare provider 1420 prescribes a colon preparation reagent a user and assigns a rectal effluent monitoring task on an application platform 1410. Consistent with the present disclosure, causing the home testing kit to be physically provided to user 1410 may include shipping the test kit to user 1410, sending an instruction to a third party to ship a test kit to user 1410, physically providing user 1410 with a test kit, or conveying a test to user 1410 in any other way. The user after consuming a bowel preparation reagent can activate the assessment on the application platform. The captured images of the rectal effluent can be automatically uploaded along with other information or can be manually uploaded by the user. An operator (such as a healthcare provider 1420, insurance company 1470, etc.) may conduct a query on data structure 1466 to identify users that meet the selected criteria. In some embodiments, user 1410 may send the message directly to healthcare provider 1420. In other embodiments, user 1410 may send the message using a dedicated application associated with rectal effluent analysis unit 1440, and the message may be conveyed to healthcare provider 1460. The message may be text or voice based or may occur as a button pushed or box checked in response to a prompt on a user interface. Alternatively, the message may simply be the scanning or entry of a code. Thereafter, healthcare provider 1420 may send a verification code to user 1410. According to one embodiment, the verification code may be sent in a text message directly to user 1410 after receiving the confirmation message or may be provided through a user interface of an application accessed via a device of user 1410. As an alternative to an exchange of electronic messages in order to obtaining the verification code, the verification code may be physically provided with the home testing kit.

Process may continue when user 1410 follows instructions associated with the specific medical examination, uses mobile communications device 1415 to capture image 1430, and transmits image data together with (or in a manner that causes it to be associated with) the verification code to medical analysis unit 1440. The image data transmitted to image analysis unit 1440 may include image 1430, a cropped image with examined object 1434, a processed version of image 1430 (e.g., one where the colour of at least part of the pixels of image 1430 was corrected based on colorized surface 1432), or data derived from image 1430.

Process may continue when the rectal effluent analysis unit 1440 determines readiness level and a score of the images obtained via image capturing device 1434, possibly taking into account local illumination conditions and/or image capturing settings effects. In other words, rectal effluent analysis unit 1440 may inspect the image of examined object 1434 after the effects of the local illumination conditions and/or of the effects of the image capturing settings are removed. In another example, rectal effluent analysis unit 1440 may inspect the image of examined object 1434 with a function that takes into account local illumination conditions and/or image capturing settings effects. The rectal effluent analysis unit 1440 may transmit the test results to healthcare provider 1420 and/or to other entities (such as user 1410, insurance company 1470, pharmacy 1480, and so forth).

In an embodiment, analysis of images of rectal effluent by the system can detect abnormal patterns or features that may indicate the presence of these conditions, such as the presence of blood or abnormal cells. In another embodiment, the system can predict the presence of certain health conditions such as colorectal cancer, inflammatory bowel disease (IBD), or infections such as Clostridioides difficile (C. diff). In another embodiment, the system can recommend further diagnostic tests or procedures, such as a colonoscopy, to confirm the diagnosis or to rule out other potential causes of the symptoms. In yet another embodiment, the system can recommend a treatment plan based on the identified health condition, taking into account the patient's medical history and other relevant factors.

In an embodiment, the artificial intelligence unit can be trained on a large dataset of images of rectal effluent, and the results of the analysis can be compared to the known diagnoses to improve the accuracy of the system over time. The feedback loop can be used to validate the results obtained by the AI system. For example, the results of the AI analysis can be compared to the results of other diagnostic tests such as a colonoscopy or biopsy, to confirm the accuracy of the AI system. The feedback loop can also be used to update the AI system's algorithm and improve its performance, based on the results of the analysis and the feedback received. The feedback loop can also be used to monitor the AI performance over time and detect any potential issues.

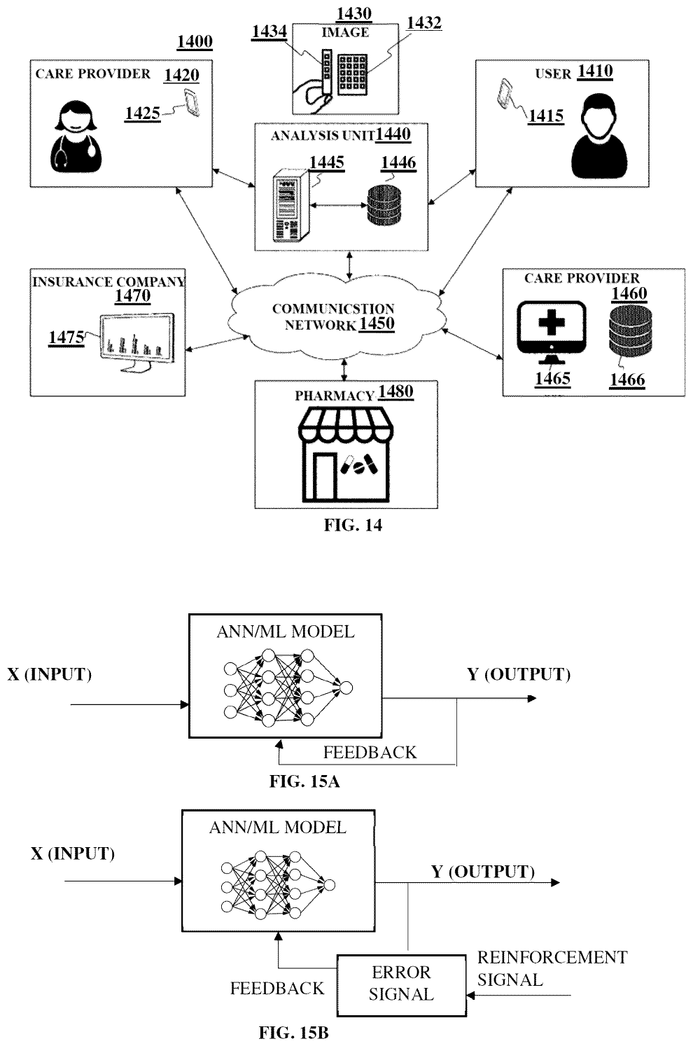

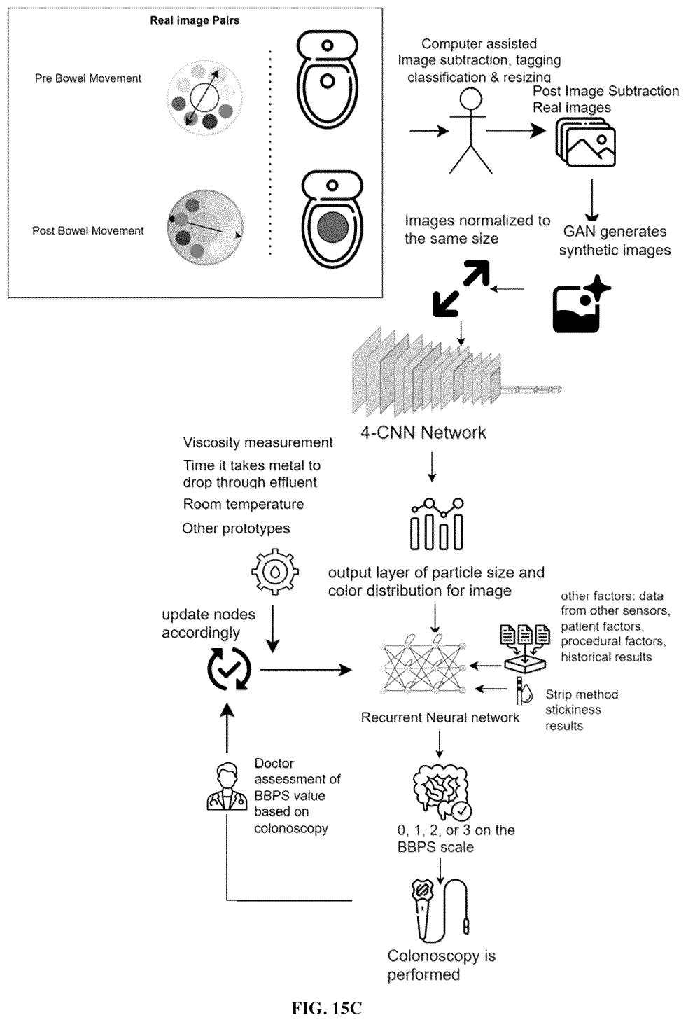

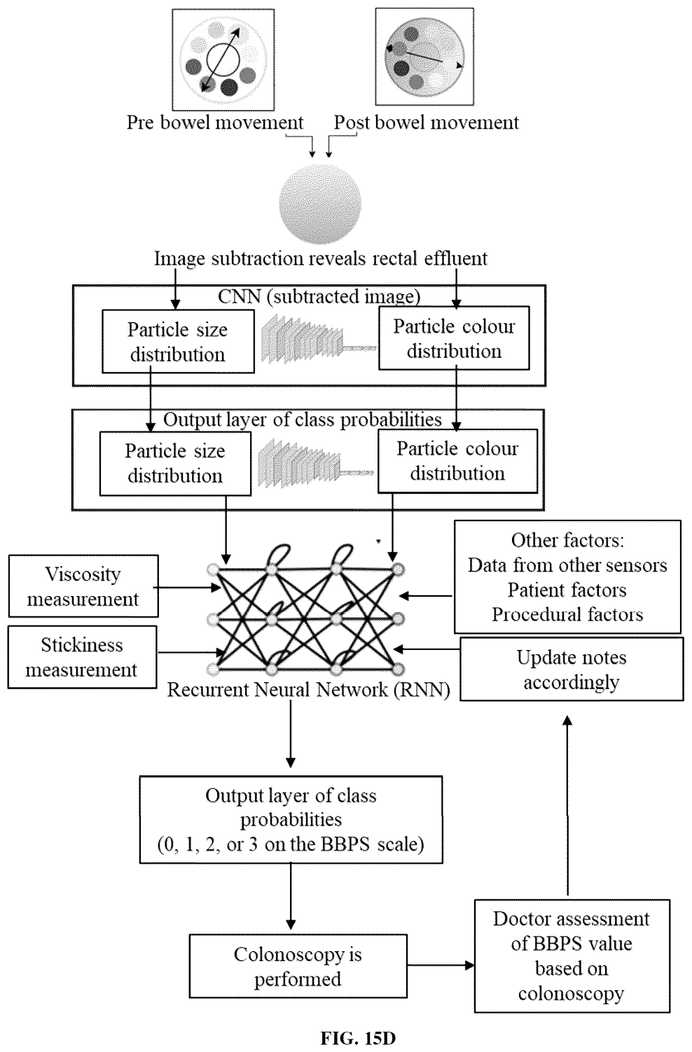

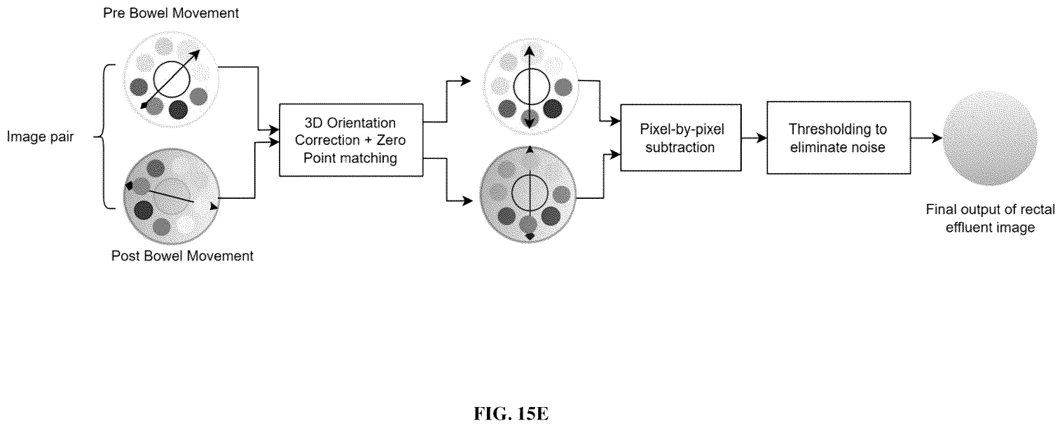

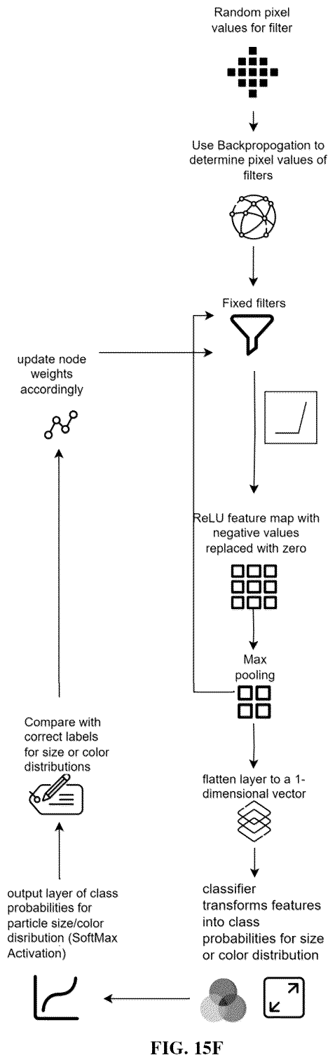

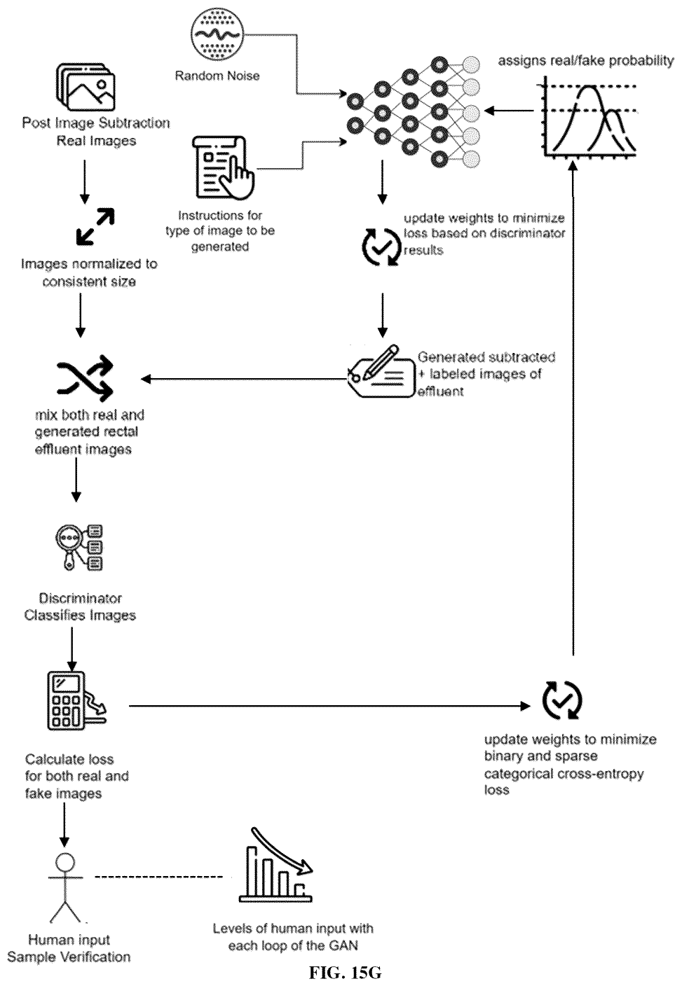

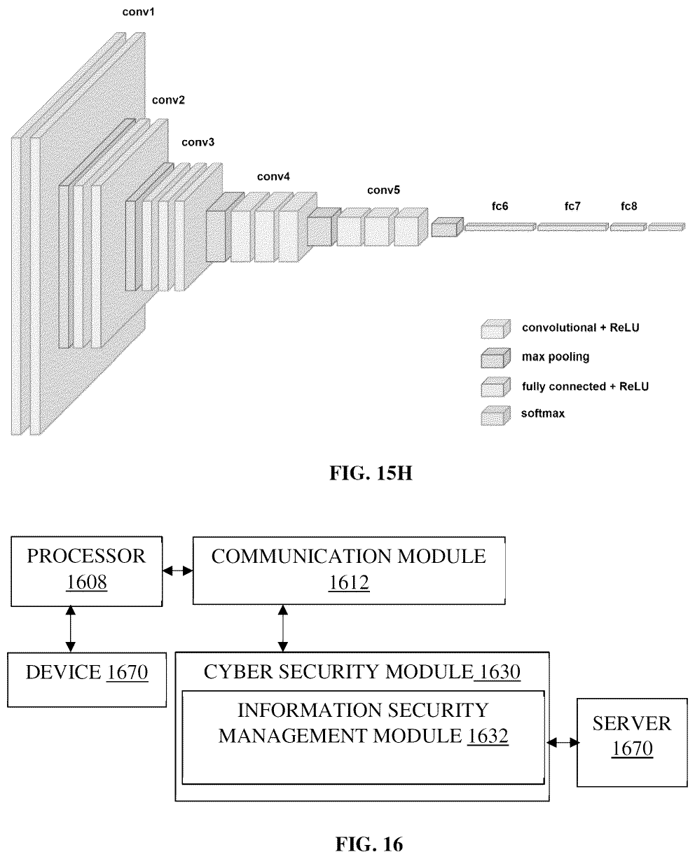

FIG. 15A shows a structure of the neural network/machine learning model with a feedback loop. Artificial neural networks (ANNs) model comprises an input layer, one or more hidden layers, and an output layer. Each node, or artificial neuron, connects to another and has an associated weight and threshold. If the output of any individual node is above the specified threshold value, that node is activated, sending data to the next layer of the network. Otherwise, no data is passed to the next layer of the network. A machine learning model or an ANN model may be trained on a set of data to take a request in the form of input data, make a prediction on that input data, and then provide a response. The model may learn from the data. Learning can be supervised learning and/or unsupervised learning and may be based on different scenarios and with different datasets. Supervised learning comprises logic using at least one of a decision tree, logistic regression, support vector machines. Unsupervised learning comprises logic using at least one of a k-means clustering, a hierarchical clustering, a hidden Markov model, and an apriori algorithm. The output layer may predict or detect a readiness level and a health issue based on the input data and can recommend a further clinical diagnosis.

In an embodiment, ANN's may be a Deep-Neural Network (DNN), which is a multilayer tandem neural network comprising Artificial Neural Networks (ANN), Convolution Neural Networks (CNN) and Recurrent Neural Networks (RNN) that can recognize features from inputs, do an expert review, and perform actions that require predictions, creative thinking, and analytics. In an embodiment, ANNs may be Recurrent Neural Network (RNN), which is a type of Artificial Neural Networks (ANN), which uses sequential data or time series data. Deep learning algorithms are commonly used for ordinal or temporal problems, such as language translation, Natural Language Processing (NLP), speech recognition, and image recognition, etc. Like feedforward and convolutional neural networks (CNNs), recurrent neural networks utilize training data to learn. They are distinguished by their “memory” as they take information from prior input via a feedback loop to influence the current input and output. An output from the output layer in a neural network model is fed back to the model through the feedback. The variations of weights in the hidden layer(s) will be adjusted to fit the expected outputs better while training the model. This will allow the model to provide results with far fewer mistakes.

The neural network is featured with the feedback loop to adjust the system output dynamically as it learns from the new data. In machine learning, backpropagation and feedback loops are used to train an AI model and continuously improve it upon usage. As the incoming data that the model receives increases, there are more opportunities for the model to learn from the data. The feedback loops, or backpropagation algorithms, identify inconsistencies and feed the corrected information back into the model as an input.

Even though the AI/ML model is trained well, with large sets of labelled data and concepts, after a while, the models' performance may decline while adding new, unlabeled input due to many reasons which include, but not limited to, concept drift, recall precision degradation due to drifting away from true positives, and data drift over time. A feedback loop keeps the AI results accurate and ensures that the model maintains its performance and improvement, even when new unlabeled data is assimilated. A feedback loop refers to the process by which an AI model's predicted output is reused to train new versions of the model.

Initially, when the AI/ML model is trained, a few labelled samples comprising both positive and negative examples of the concepts (e.g., images of a rectal effluent) are used that are meant for the model to learn. Afterward, the model is tested using unlabeled data. By using, for example, deep learning and neural networks, the model can then make predictions on whether the desired concept/s (for e.g., the readiness level for a clinical procedure) are in unlabeled images. Each image is given a probability score where higher scores represent a higher level of confidence in the models' predictions. Where a model gives an image a high probability score, it is auto labelled with the predicted concept. However, in the cases where the model returns a low probability score, this input may be sent to a controller (maybe a human moderator) which verifies and, as necessary, corrects the result. The human moderator may be used only in exception cases. The feedback loop feeds labelled data, auto-labelled or controller-verified, back to the model dynamically and is used as training data so that the system can improve its predictions in real-time and dynamically.

FIG. 15B shows a structure of the neural network/machine learning model with reinforcement learning. The network receives feedback from authorized networked environments. Though the system is similar to supervised learning, the feedback obtained in this case is evaluative, not instructive, which means there is no teacher as in supervised learning. After receiving the feedback, the network performs adjustments of the weights to get better predictions in the future. Machine learning techniques, like deep learning, allow models to take labeled training data and learn to recognize those concepts in subsequent data and images. The model may be fed with new data for testing, hence by feeding the model with data it has already predicted over, the training gets reinforced. If the machine learning model has a feedback loop, the learning is further reinforced with a reward for each true positive of the output of the system. Feedback loops ensure that AI results do not stagnate. By incorporating a feedback loop, the model output keeps improving dynamically and over usage/time.

Referring to FIG. 15C, it shows a workflow for analyzing bowel preparation quality using a combination of image processing techniques and neural networks. The process begins with capturing real image pairs before and after a bowel movement. These images undergo computer-assisted image subtraction to highlight differences. The subtracted images are then tagged, classified, and resized for consistency. Following this, Generative Adversarial Networks (GANs) are used to generate synthetic images, which are normalized to the same size as the real images. The normalized images are fed into a Convolutional Neural Network (CNN) that processes the images to extract features related to particle size and color distribution. The output layer of CNN produces detailed information on these features. This output, along with additional data such as stickiness, viscosity, and other relevant factors, is fed into a Recurrent Neural Network (RNN) to analyze temporal dependencies. The RNN then calculates a Boston Bowel Preparation Scale (BBPS) score, which ranges from 0 to 3. This score helps in deciding whether a colonoscopy should be performed. The doctor's assessment of the BBPS value, based on the colonoscopy results, is used to update the network's nodes, refining its accuracy and performance for future assessments.

Referring FIG. 15D, it shows imaging and analysis process using Convolutional Neural Networks (CNNs) and Recurrent Neural Networks (RNNs) for bowel preparation analysis. The process begins with the acquisition of two sets of images: one taken before a bowel movement (pre), and one taken after (post). A pixel-by-pixel subtraction is performed between the pre and post images to reveal the fractal effluent, highlighting the changes due to the bowel movement. The subtracted image is then analyzed using a CNN, which has been trained to identify and classify particle size distribution and particle color distribution within the effluent. The CNN provides an output layer of class probabilities for both particle size distribution and particle color distribution, indicating the likelihood of each class being present in the image. An RNN takes the class probabilities from the CNN's output layer and integrates them with additional data such as viscosity measurements, stickiness measurements, and other sensor data, along with patient and postural factors. The RNN generates an output layer of class probabilities corresponding to the BBPS (Boston Bowel Preparation Scale) values of 0, 1, 2, or 3. This scale assesses the cleanliness of the colon. If the analysis suggests that the colon is adequately prepared, a further medical procedure, such as a colonoscopy, may be performed. The BBPS value obtained from the colonoscopy is used for assessment. The software platform updates the neural network nodes based on the BBPS score generated, refining the model for future analyses. This sophisticated process combines image processing techniques with machine learning models to provide a comprehensive analysis of bowel movement quality, aiding medical professionals in determining the readiness of the colon for procedures like colonoscopies.

Image subtraction is a method used to highlight changes between two images taken before and after a bowel movement.

Referring to FIG. 15E, initially, both images undergo preparation to ensure consistency: they depict the same scene with known particle distributions, sizes, and colors. To align them accurately in three-dimensional space, corrections such as rotation, translation, and scaling are applied using specialized software like MATLAB or Python. The images are further adjusted for color intensity and white balance to standardize the appearance of key elements of the reticle. Subtraction begins by comparing pixel values between the pre- and post-bowel movement images. Each pixel in the ‘before’ image is subtracted from its corresponding pixel in the ‘after’ image, and the absolute value of these differences is taken to ensure positivity. This process effectively isolates changes caused by rectal effluent, producing an image where these changes appear prominently. To refine the resulting image and eliminate irrelevant details, various thresholding techniques are applied. Global thresholding sets a uniform threshold across the image, while adaptive thresholding computes thresholds based on local pixel values, adjusting for varying lighting conditions. Otsu's thresholding, an automatic method, optimizes the threshold value to minimize variance within the image. The final output is a clear depiction of rectal effluent, emphasizing the changes while minimizing noise and irrelevant elements. This method ensures that only significant alterations between the pre- and post-bowel movement images are highlighted, facilitating detailed analysis and diagnosis in medical contexts.

In one embodiment, a Generative Adversarial Network (GAN) is employed to create training images for a Convolutional Neural Network (CNN). The GAN consists of two neural networks—the generator and the discriminator—that are trained concurrently through an adversarial process. This training loop involves several key steps: In step 1, the generator creates new images using a 2D convolutional network and random noise, along with specific instructions regarding the type of image to be generated. In step 2, the discriminator evaluates both real and generated images, performing a binary classification to distinguish real from fake images. It then classifies real and fake images into specific categories, such as BPPS scale classes for rectal effluent images. In step 3, losses are calculated for both the discriminator and the generator. For binary classification, a binary cross-entropy loss is computed, averaging the losses for real and fake images. For multiclass classification, a sparse categorical cross-entropy loss is calculated, again averaging the losses for real and fake images. In step 4, the weights of both networks are updated to minimize the respective losses. This is achieved using a weight update function that adjusts the network parameters based on the calculated losses. In step 5, human verification is incorporated at various stages to ensure the quality and accuracy of the generated images. The level of human input can vary with each iteration of the GAN training loop. This process is iterated multiple times. With each iteration, the generator improves its ability to produce high-quality, realistic images, while the discriminator becomes more adept at distinguishing real images from fake ones.

Referring to FIG. 15F, the flow chart illustrates the process for generating training images. Real images are captured and processed to remove background or unwanted elements through image subtraction. The images are then normalized to a consistent size for uniformity before being input into the network. The generator receives normalized images and random noise, producing labeled images of rectal effluent that are mixed with real images. The discriminator then performs binary and multiclass classification, calculating and updating losses accordingly. Human input is utilized for sample verification, ensuring the generated images meet quality standards. Instructions for particle size and color distribution are provided to guide the GAN. The process repeats, continually enhancing the generator's capability to create indistinguishable images and the discriminator's classification accuracy. Through this adversarial training process, the generator eventually produces images that the discriminator classifies as real, effectively creating high-quality training images for CNN.

Referring to FIG. 15G, the learning process of a Convolutional Neural Network (CNN) to recognize and classify features in images begins with feature extraction using fixed filters. Filters such as Canny edge detection, Sobel filter, or Gaussian filter are applied to the input image to create feature maps that highlight important features. Canny edge detection identifies edges by detecting sharp changes in intensity or color, while the Sobel filter focuses on the gradient and direction of light intensity to detect edges. The Gaussian filter smooths and blurs the image to remove noise and detail.

While primitive methods use hand-engineered filters, CNNs have the ability to learn these filters through training. Initially, the pixel values of the filters are set randomly. Using backpropagation, these values are adjusted to minimize the loss function, thereby learning the optimal values for detecting features in the images. The Rectified Linear Unit (ReLU) activation function is then applied to the feature maps, replacing negative values with zero to introduce non-linearity, allowing the network to solve more complex problems.

Next, a down sampling operation such as max pooling is used to reduce the spatial dimensions (width and height) of the input volume for the next convolutional layer. Max pooling takes the largest element from the rectified feature map within a certain window (usually 2×2), making feature detection invariant to scale and orientation changes. The output of the pooling layer is then flattened into a single vector, transforming the 2D matrix data into a 1D feature vector, which is fed into the fully connected layer.

The flattened feature vector is passed through one or more fully connected layers where the actual classification takes place. These layers transform the features learned during the CNN process into final outputs such as class scores. In the output layer, the softmax activation function is applied to convert the output into a probability distribution over the predefined classes. For example, in the context of particle size and color distribution, the softmax layer provides probabilities for each class, indicating the likelihood of the input image belonging to a particular class.

The predicted probabilities are then compared with the true labels. The difference between the predicted and true labels is calculated using a loss function, and through backpropagation, the weights of the nodes are updated to minimize this loss, refining the network's accuracy. This entire process, from applying fixed filters to updating weights, enables CNN to learn and accurately classify features in images, improving its performance through iterative training.

Referring to FIG. 15H, the CNN architecture for image recognition typically consists of a sequence of layers designed to effectively capture and classify image features. The process begins with convolutional layers, which apply various filters to the input image to extract key features. These layers are paired with Rectified Linear Unit (ReLU) activations, which introduce non-linearity by replacing negative values with zero, enabling the network to model more complex patterns. Following the convolutional layers, max pooling layers are used for dimensionality reduction. Max pooling selects the maximum value from a specified window, reducing the spatial dimensions of the feature maps and making the network more robust to variations in scale and orientation.

The reduced-dimensional feature maps are then flattened into a single vector and passed through fully connected layers, which perform pattern recognition by learning the relationships between the extracted features. Finally, a softmax layer is used for classification, converting the output into a probability distribution over predefined class. This comprehensive design allows CNNs to effectively capture spatial and temporal dependencies in images, making them highly effective for tasks such as image and video recognition, image classification, and other related applications.

In an embodiment, the system may comprise a cyber security module.

In an embodiment, the cyber security module further comprises an information security management module providing isolation between the system and the server.

FIG. 16 shows the block diagram of the cyber security module. The communication of data between the system 1600 and the server 1670 through the communication module 1612 is first verified by the information security management module 1632 before being transmitted from the system to the server or from the server to the system. The information security management module is operable to analyze the data for potential cyber security threats, to encrypt the data when no cyber security threat is detected, and to transmit the data encrypted to the system or the server.

In one aspect, a secure communication management (SCM) computer device for providing secure data connections is provided. The SCM computer device includes a processor in communication with memory. The processor is programmed to receive, from a first device, a first data message. The first data message is in a standardized data format. The processor is also programmed to analyze the first data message for potential cyber security threats. If the determination is that the first data message does not contain a cyber security threat, the processor is further programmed to convert the first data message into a first data format associated with the vehicle environment and transmit the converted first data message to the vehicle system using a first communication protocol associated with the vehicle system.

According to an embodiment, secure authentication for data transmissions comprises provisioning a hardware-based security engine (HSE) located in communications system, said HSE having been manufactured in a secure environment and certified in said secure environment as part of an approved network; performing asynchronous authentication, validation and encryption of data using said HSE, storing user permissions data and connection status data in an access control list used to define allowable data communications paths of said approved network, enabling communications of the communications system with other computing system subjects to said access control list, performing asynchronous validation and encryption of data using security engine including identifying a user device (UD) that incorporates credentials embodied in hardware using a hardware-based module provisioned with one or more security aspects for securing the system, wherein security aspects comprising said hardware-based module communicating with a user of said user device and said HSE.

In an embodiment, the cyber security module further comprises an information security management module providing isolation between the system and a server. The information security management module is operable to receive data from the communication module, exchange a security key at a start of a communication between the communication module and the server, receive the security key from the server, authenticate an identity of the server by verifying the security key, analyze the security key for a potential cyber security threat, negotiate an encryption key between the communication module and the server, encrypt the data, and transmit the encrypted data to the server when no cyber security threat is detected. The information security management module is further operable to exchange a security key at a start of a communication between the communication module and the server, receive the security key from the server, authenticate an identity of the server by verifying the security key, analyze the security key for a potential cyber security threat, negotiate an encryption key between the system and the server, receive encrypted data from the server, decrypt the encrypted data, perform an integrity check of the decrypted data, and transmit the decrypted data to the communication module when no cyber security threat is detected.

### INCORPORATION BY REFERENCE

All references, including granted patents and patent application publications, referred herein are incorporated herein by reference in their entirety.
U.S. Pat. No. 11,087,467B2 titled “Systems and methods for urinalysis using a personal communications device.”;
WO2021146420A1 titled “Systems and methods for assessing colonoscopy preparation”;
WO2021260663A1 titled “Toilet commode with stool examination device”;
US20160128514A1 titled “Motor unit, tool unit and hand blender”;
US20070014187A1 titled “Whisk-a-bowl”;
U.S. Pat. No. 8,454,223B2 tiled “Blender arm and food processor”;
U.S. Pat. No. 10,717,060B2 titled “Programmable wireless control system for mixers”;
U.S. Pat. No. 9,125,518B2 titled “Remote controlled food processor”;
U.S. Pat. No. 6,394,640B1 titled “Mixing accessory having flexible wires mounted in a crossed manner”;
US20120257471A1 titled “Beater assembly and kitchen appliance with a beater assembly”;
US20070159917A1 titled “Pivoting handheld food preparation appliance and associated method”;
US20220211354A1 titled “Biomonitoring devices, methods, and systems for use in a bathroom setting”;
U.S. Pat. No. 9,756,297B1 titled “Camera for viewing and sensing the health of a user sitting on a toilet”;
U.S. Pat. No. 9,498,583B2 titled “Froth detection system and method”.

1-184. (canceled)

185. A method comprising:
providing a reference background for measuring a plurality of parameters;
capturing an image of rectal effluent along with the reference background;
assessing the image for readiness level of bowel preparation;
estimating the readiness level by measuring the plurality of parameters; and
providing an assessment of bowel preparation to undergo a medical procedure.

186. The method of claim 185, wherein the reference background comprises a reticle wherein the reticle is used for the measuring at one or more of the plurality of parameters.

187. The method of claim 185, wherein the plurality of parameters comprises two or more of color, turbidity, particulate concentration, size distribution, stickiness, and viscosity.

188. The method of claim 185, wherein the image comprises one or more images and videos.

189. The method of claim 185, wherein assessing comprises analysis of the image.

190. The method of claim 185, wherein the readiness level comprises a cleanliness class comprising one of an unacceptable colon state, a poor colon state, a fair colon state, and a good colon state.

191. A device comprising:
a disposable assembly comprising an adjustable frame and an endoscope; and
a reusable assembly comprising a detachable Universal Serial Bus port cable configured to connect with an external device.

192. The device of claim 191, wherein the device is affixable to a toilet commode.

193. The device of claim 191, wherein the adjustable frame comprises 3D target for aiding in focusing and is configured for providing sharp and clear image.

194. The device of claim 193, wherein the 3D target comprises a reticle.

195. The device of claim 194, wherein the reticle acts as a reference point for measuring one or more of a color measurement, a diameter measurement of a microscopic object, a thickness measurement of the microscopic object, calibration and alignment.

196. The device of claim 195, wherein the endoscope is configured to capture an image of a suspension in a toilet commode.

197. The device of claim 196, wherein the endoscope comprises a light emitting diode.

198. A system comprising an image capturing device comprising a reference background configured to be affixable to a toilet commode, a communication module, a processor and a non-transitory storage medium comprising instructions that, when executed, causes the processor to:
capturing an image of the rectal effluent along with the reference background;
assessing the image for readiness level of bowel preparation;
estimating the readiness level by measuring a plurality of parameters; and
providing an assessment of bowel preparation to undergo a medical procedure.
